(19) United States
(12) Patent Application Publication
(10) Pub. No.: US 2008/0081039 A1
(43) Pub. Date: Apr. 3, 2008

(54) Inorganic Selenium For Treatment Of Cancer

(75) Inventors: Niall Corcoran, Flemington (AU); Christopher Hovens, Surry Hills (AU); Anthony Costello, Port Melbourne (AU)

Correspondence Address: KNOBBE MARTENS OLSON & BEAR LLP, 2040 MAIN STREET, FOURTEENTH FLOOR, IRVINE, CA, 92614, US

(73) Assignee: Velacor Therapeutics Pty. Ltd., Parkville (AU)

(21) Appl. No.: 11/575,743

(22) PCT Filed: Jan. 31, 2005
(86) PCT No.: PCT/AU05/00111

§ 371 Date: Jun. 7, 2007

Related U.S. Application Data
(60) Provisional application No. 60/611,793, filed on Sep. 21, 2004.

(30) Foreign Application Priority Data
Sep. 21, 2004 (AU) 2004905422

(51) Int. Cl. A61K 39/395; A61K 33/04; A61P 35/00; C12N 5/00; A61K 33/24

(52) U.S. Cl. 424/138.1; 424/617; 424/702; 435/375

(57) ABSTRACT

The present invention discloses the use of selenate or its pharmaceutically acceptable salts, especially in supranutritional amounts, in methods and compositions for inhibiting the growth or proliferation of tumor cells. The present invention also discloses the use of selenate or its pharmaceutically acceptable salts in combination with one or both of a hormone ablation therapy and a cytostatic agent or cytotoxic agent, for inhibiting the growth or proliferation of tumor cells. In certain embodiments, the methods of the invention are useful for treating or preventing cancers, especially cancers in which the Akt signaling pathway is activated, such as prostate cancer. Additionally, the present invention discloses the use of selenate or its pharmaceutically acceptable salts in combination with a hormone-ablation therapy and optionally a cytostatic agent or cytotoxic agent in methods and compositions for treating hormone- dependent cancers.

### FIELD OF THE INVENTION

This invention relates generally to the use of selenate or its pharmaceutically acceptable salts, especially in supranutritional amounts, in methods and compositions for inhibiting the growth or proliferation of tumor cells. The present invention also relates to the use of selenate or a pharmaceutically acceptable salt thereof in combination with at least one of a hormone ablation therapy, a cytostatic agent or cytotoxic agent, for inhibiting the growth or proliferation of tumor cells. In certain embodiments, the methods of the invention are useful for treating or preventing cancers, especially cancers in which the Akt signaling pathway is activated, such as prostate cancer. Additionally, the present invention relates to the use of selenate or a pharmaceutically acceptable salt thereof in combination with a hormone-ablation therapy and optionally a cytostatic agent or a cytotoxic agent in methods and compositions for treating hormone-dependent cancers.

### BACKGROUND OF THE INVENTION

There has been much interest in the use of selenium compounds as cancer-preventive agents. Studies spanning the last 20 years have documented their cancer-preventive action in tumors of the mammary gland (Ip, C. 1981, Cancer Res. 41:4386-4390), colon (Reddy et al. 1981, Cancer Res. 47:5901-5904), lung and prostate (Clark et al. 1996, Jama 276:1957-1963). Both phase II and III clinical trials for prostate cancer prevention using an organic selenomethionine are currently underway (Nelson et al. 1999, supra).

Selenium compounds used in chemoprevention studies can broadly be classified into inorganic and organic selenium forms. The typical form of inorganic selenium, sodium selenite, (Na₂SeO₃) is relatively toxic, causing single- and double-strand break DNA damage, whilst the typical organic selenium entity, selenomethionine (SeMet) is relatively non-toxic and non-DNA-damaging (Lu et al. 1995, supra; Sinha et al. 1996, supra; Stewart et al. 1999, supra).

Organic selenomethionine, the major constituent of dietary selenium, is incorporated into cellular selenoproteins, such as thioredoxin reductase and glutathione peroxidase via a series of complex intermediates (Allan et al. 1999, Annu. Rev. Nutr. 19:1-16). Since these selenocysteine-containing selenoenzymes, such as the antioxidant glutathionine peroxidase and the redox-regulatory thioredoxins, are involved in cellular responses to mutagenic oxidative stress, it has long been proposed that supranutritional levels of selenium intake boosts anticarcinogenic cellular activity by promoting a cellular reducing environment (Allan et al. 1999, supra).

It is, however, becoming increasingly clear that the nature of the anti-tumorigenic action of selenium compounds is dependent on the chemical form in which the element is administered (IP, C. 1998, J. Nutr. 128:1845-1854). Organic selenium in the form of methylseleninic acid induces G1 arrest, as well as DNA fragmentation and caspase mediated cleavage of PARP, which are two markers of apoptosis, in DU145 prostate cancer cells (Jiang et al. 2001, Cancer Res. 61:3062-3070).

In contrast, selenite induces S-phase arrest and apoptotic DNA fragmentation, which is independent of caspase function (Jiang et al. 2002, Mol. Cancer Ther. 1:1059-1066). Differences have also been reported for the inorganic selenium compounds, with selenite and selenate inhibiting lymphocyte growth via different mechanisms (Spyrou et al. 1996, Cancer Res. 56:4407-4412). These differences may be explained in part by variations in metabolism and bioavailability between in vitro cell culture and in vivo studies (Dong et al. 2003, Cancer Res. 63:52-59).

Hormone-dependent cancers include tumor cells having hormone receptors and the growth and proliferation of these tumor cells is encouraged by the presence of the hormone. Hormone ablation therapy, either surgical or chemical, is used to halt, at least for a period of time, the growth and proliferation of tumor cells in hormone-dependent tumors such as prostate, testicular, thyroid, breast, ovarian and uterine cancers.

Prostate cancer is a prevalent cancer in human males and treatment of patients with advanced prostate carcinoma growth typically involves medical or surgical castration (Huggins, C. and Hodges, C. V. 1941, Cancer Res. 1:293-297). Up to 80% of patients demonstrate a temporary response lasting a median of 12-18 months, before continued tumor growth is evident despite castrate levels of testosterone (Petrylak, D. P. 1999, Urology 54:30-35). Once androgen-independent growth is established, median life expectancy is 9-12 months. While traditional chemotherapy can improve pain scores and augment quality of life, it does not offer any significant survival benefit.

The molecular mechanisms underlying the transition to androgen independent growth are incompletely understood. Changes in the androgen receptor signaling may play a role, including upregulation of receptor expression, promiscuous ligand binding as well as ligand independent receptor transactivation by other growth signaling cascades (Feldman, B. J. and Feldman, D. 2001, Nat. Rev. Cancer 1:34-45).

Concomitantly, prostate cancer cells acquire a number of mechanisms that protect from induced cell death, explaining in part their chemoresistance (Gurumurthy et al. 2001, Cancer Metastasis Rev. 20:225-243). The role of the PI3K/Akt pathway in this is increasingly recognized. Akt is a serine/threonine kinase that is activated in response to membrane receptor stimulated PI3K phosphorylation. Akt, in turn, regulates the activity of several proteins involved in the control of apoptosis, including the Forkhead (FKHR) transcription factors, Bad, Caspase 9, GSK-3β and mTOR (Vivanco, I. and Sawyers, C. L. 2002, Nat. Rev. Cancer 2:289-501). Akt overactivity is common in androgen-independent prostate cancer, often as a result of PTEN hypofunction, a phosphatase that inactivates PI3K, and is sufficient to cause androgen-independent growth (Whang et al. 1998, Proc. Natl. Acad. Sci. USA 95:5246-5250).

Radiotherapy resistance in tumor cells has also been linked with upregulation of the PI3K/Akt pathway (Soderlund et al. 2005, Int. J. Oncol., 26:25-32; Zhan et al. 2004, Histol. Histopathol., 19:915-923; Tanno et al. 2004, Cancer Res., 64:3486-3490; Li et al. 2004, Oncogene, 23:4594-4602; McKenna et al., 2003, Genes Chromosomes Cancer, 28:330-338; Liang et al. 2003, Mol. Cancer Ther. 2:353-360).

Interest in the clinical use of selenium-containing compounds as a chemopreventive agent has become widespread following the publication of Clark et al. (1996, supra). These results have spurred a number of human clinical trials using supranutritional selenomethionine as a chemopreventive agent for prostate cancer (Meuillet et al. 2004, J. Cell Biochem. 91:443-458). However, given the relatively short intervention time compared to the long natural history of prostate cancer, it is possible that rather than preventing the transformation of normal prostate epithelia to neoplasia, selenium more likely inhibits the growth of malignant cells (Corcoran et al. 2004, J. Urol. 171:907-910). Evidence is accumulating that the anticancer activity of selenium is not related to the element per se but is dependent upon its chemical form (Menter et al. 2000, Cancer Epidemiol. Biomarkers Prev. 9:1171-1182; Jiang et al. 2002, supra; Kim et al. 2003, Biochem. Pharmacol. 66:2301-2311). The mechanism for this activity is also unclear.

However, it has been noted that there are marked differences in tumor progression of human prostate tumor cells in vivo, depending upon the selenium compound administered. For instance, inorganic sodium selenate significantly reduces tumor progression in comparison to organic selenomethionine, methylselenocysteine or selenized yeast (Corcoran et al. 2004, supra). In addition, certain selenium compounds, such as sodium selenite have been found to be toxic to cells when used at certain doses (Jiang et al. 2002, supra).

Therefore, there is a need to identify suitable forms of selenium compounds to treat or prevent cancer growth. In particular, there is a need to understand the selective underlying molecular mechanisms of action of various selenium compounds on cancer cells with a view to designing more effective therapeutic regimens.

### SUMMARY OF THE INVENTION

The present invention is predicated in part on the discovery that a specific type of inorganic selenium compound, namely selenate, significantly inhibits tumor cell proliferation including the proliferation of hormone-independent tumor cells and hormone-dependent tumor cells, especially when used at high or supranutritional amounts, as compared to other selenium compounds. It has also been found that selenate and its pharmaceutically acceptable salts have an inhibitory effect on tumor cells, especially prostate tumor cells, in which the Akt signaling pathway is activated, and have a strong synergistic inhibitory effect on tumor cell growth when used in combination with at least one of a cytostatic agent, a cytotoxic agent and a radiotherapy that is optionally administered with a radiosensitizing agent. Further, it has been found that selenate and its pharmaceutically acceptable salts have an inhibitory effect on hormone-dependent tumor cell growth when used in combination with a hormone ablation therapy and optionally one or more of a cytostatic agent, a cytotoxic agent and a radiotherapy that is optionally administered with a radiosensitizing agent.

Accordingly, in one aspect, the present invention provides methods for inhibiting the growth or proliferation of tumor cells in which the Akt signaling pathway is activated. These methods generally comprise exposing the tumor cells to an Akt signaling pathway activation-inhibiting amount of selenate or a pharmaceutically acceptable salt thereof. In some embodiments, the activation of the Akt signaling pathway involves activation of at least one member selected from Akt, mTOR, GSK-3β and FKHR. In illustrative examples of this type, the activation of the Akt signaling pathway involves phosphorylation of Akt (e.g., phosphorylation of the Thr 308 and Ser 473 residues of Akt). In other embodiments, the activation of the Akt signaling pathway involves inactivation of PTEN. In some embodiments, the Akt signaling pathway is over-activated. In some embodiments, the amount of selenate or its pharmaceutically acceptable salt, to which the tumor cells are exposed, is a supranutritional amount. Suitably, the tumor cells are selected from oral squamous cell carcinoma, thyroid cancer, hepatocellular carcinoma, prostate carcinoma, fibrosarcoma, ovarian carcinoma, uterine or endometrial cancer, pancreatic carcinoma, stomach cancer, breast cancer, lung cancer, renal cell carcinoma, colon cancer, melanoma, acute leukemia and brain cancer (eg: astrocytoma and glioblastoma). In specific embodiments, the tumor cells are prostate cancer cells, including hormone-independent prostate cancer cells.

In another aspect, the present invention provides methods for treating cancer, especially a hormone-independent cancer or a hormone-dependent cancer, in a subject. These methods generally comprise administering to the subject a therapeutically effective amount of selenate or a pharmaceutically acceptable salt thereof In some embodiments, the therapeutically effective amount is a supranutritional amount. In specific embodiments, the cancer is prostate cancer, especially a hormone-independent or a hormone-dependent prostate cancer. Thus, in a related aspect, the invention provides methods for treating prostate cancer, especially a hormone-independent or a hormone-dependent prostate cancer, comprising administering to a subject in need of such treatment a therapeutically effective amount of selenate or a pharmaceutically acceptable salt thereof.

In yet another aspect, the present invention provides methods for inhibiting hormone-dependent growth of tumor cells, comprising exposing the tumor cells to a hormone-dependent tumor cell growth-inhibiting amount of selenate or a pharmaceutically acceptable salt thereof and a hormone ablation therapy.

In still another aspect, the present invention provides methods for treating a hormone-dependent cancer in a subject, comprising administering a therapeutically effective amount of selenate or a pharmaceutically acceptable salt thereof in combination with a hormone ablation therapy. The hormone-dependent cancers are suitably selected from androgen-dependent cancers and estrogen-dependent cancer. In certain embodiments, the hormone-dependent cancer is an androgen-dependent cancer such as prostate cancer. In some embodiments, the hormone-dependent cancer is selected from prostate cancer, testicular cancer, breast cancer, ovarian cancer, uterine cancer, endometrial cancer, thyroid cancer and pituitary cancer.

In a further aspect, the present invention provides a use of selenate or a pharmaceutically acceptable salt thereof in the manufacture of a medicament for treating a cancer in which the Akt signaling pathway is activated.

In still a further aspect, the present invention provides a use of selenate or a pharmaceutically acceptable salt thereof in the manufacture of a medicament for treating a cancer in which the Akt signaling pathway is activated, wherein the cancer is other than a cancer selected from PC-3 prostate cancer, 3B6 lymphoma, BL41 lymphoma and HTB123/DU4473 mammary tumor.

In yet a further aspect, the present invention provides a use of selenate or a pharmaceutically acceptable salt thereof in the manufacture of a medicament for treating a hormone-dependent cancer, wherein the selenate or its pharmaceutically acceptable salt is formulated for administration in combination with hormone ablation therapy.

In still another aspect, the present invention provides pharmaceutical compositions for treating or preventing cancer. The compositions generally comprise a supranutritional amount of selenate or a pharmaceutically acceptable salt thereof and a pharmaceutically acceptable carrier.

In some embodiments of the methods and uses broadly described above, the selenate or its pharmaceutically acceptable salt is administered in combination with at least one of a cytostatic agent, a cytotoxic agent or a radiotherapy that is optionally administered with a radiosensitizing agent. In other embodiments, the selenate is formulated in a composition with at least one cytostatic agent or cytotoxic agent. In still other embodiments, the selenate is formulated in a composition with a radiosensitizing agent for use in combination with radiotherapy.

### BRIEF DESCRIPTION OF THE DRAWINGS

FIG. 1 is a graphical and photographic representation showing that sodium selenate inhibits prostate tumor cell proliferation in an orthotopic mouse model. 6-week old male BALB/c nude mice were injected in the dorsolateral prostate with 1×10⁶ PC-3 cells. Ten animals per group then received either 5 ppm sodium selenate (NaSe) or no treatment (Con) in the drinking water for 5 weeks. Animals were then culled. (A) FIG. 1(A) graphically indicates that the weight of tumor-containing prostate glands was reduced in the mice that received sodium selenate. (B) FIG. 1(B) graphically indicates that tumor volumes, measured with a Vernier caliper, were slightly reduced (but not significantly) in mice that received sodium selenate. (C) The retroperitonium was then explored under magnification cephadally to the level of the renal vein and lymph nodes (L.N.no) greater than 0.5 mm were counted. FIG. 1(C) indicates the number of lymph nodes greater than 0.5 mm were reduced in mice that received sodium selenate. Results depict the means +/− SE. FIGS. 1(A) and 1(C) have p<0.05 vs. control. (D) FIG. 1(D) provides a compilation of BrdU and Tunel positive cell nuclei from prostate tumor samples. Results represent the means +/−SE per High power field for 4 tumor samples from the no treatment (Con) and sodium selenate (NaSe) groups. (E) FIG. 1(E) provides a representative immunohistochemical sample of BrdU positive nuclei from no treatment (control) and sodium selenate (NaSe) prostate tumor tissue samples, at ×100 magnification.

FIG. 2 is a graphical and photographic representation showing that sodium selenate inhibits proliferation of prostate carcinoma cells by inducing a G1 cell cycle block. (A) 2×10⁵ PC-3 cells were seeded in a 6-well plate and incubated for 16 hours. Sodium selenate at the indicated concentrations was then added and incubation continued for the indicated time points. Non-adherent and adherent cell fractions were then harvested and pooled and total viable cell numbers were counted by Trypan Blue Exclusion assay. FIG. 1A) graphically indicates the effect of different concentrations of sodium selenate on viable cell numbers. (B) 2.5×10⁴ PC-3 cells were seeded in a 24-well plate and 16 hours later exposed to 0.05, 0.1, or 0.25 mM sodium selenate for 36 hours, then BrdU/FrdU proliferating cell labeling reagent (Roche) added for a further 16 hours. Cells were washed, fixed and stained for BrdU/FrdU nuclear incorporation, and DAP1 for identification of cell nuclei. Results are shown in FIG. 2(B). (C) 2×10⁵ PC-3 cells were seeded in a T25 flask and incubated for 16 hours. Cells were then washed and incubated in serum-free media for a further 48 hours. Cells were then restimulated with fresh serum (FCS, 10%) containing 0.0, 0.1, 0.25, or 0.5 mM sodium selenate for a further 24 or 48 hours. Cells were then harvested, fixed and stained with proprium iodide, and FACS analysis performed. The graphical plot of mean percentages of cells in the G1 and G2/M phases of the cell cycle for three independent experiments are shown in FIG. 2(D). (E) Cell proliferation was assessed by the MTT assay. 1×10⁴ PC-3 cells were seeded in a 96-well plate and incubated for 16 hours. Indicated concentrations of sodium selenate or selenomethionine were then added and 73 hours later cell proliferative index measured by the MTT assay. The results are shown in FIG. 2(E). For all results, values shown represent the mean +S.D. from at least three independent experiments.

FIG. 3 is a photographic representation showing that sodium selenate induces upregulation of cell cycle inhibitory proteins and dephosphorylation of the retinoblastoma protein. (A) 7.5×10⁵ PC-3 cells were seeded in 6 cm dishes, then 16 hours later asynchronously growing cells were treated with 0.5 mM sodium selenate for the times indicated, then cells lysed in ELB buffer, and equal amounts of whole cell lysates (75 μg) separated on 12% SDS-PAGE gels and membranes probed with the indicated primary antibodies. The results are shown in FIG. 3(A). (B) 5×10⁵ PC-3 cells were seeded in a 6-well plate, incubated for 10 hours washed, and serum starved for 16 hours, then treated with sodium selenate (0.5 mM) for the time points shown. Cells were lysed in ELB buffer and whole cell lysates (75 μg) loaded on 10% SDS-PAGE gels and membranes probed with the indicated antibodies. The results are shown in FIG. 3(B). (C) 2×10⁵ PC-3 cells were seeded in a 6-well plate, (incubated for 16 hours then treated with sodium selenate (Na₂SeO₄, 0.5 mM) or selenomethionine (SeMet, 0.5 mM)) for the indicated times and whole cell lysates (75 μg) run out on 12.5% SDS-PAGE gels and membranes probed with the indicated antibodies. The results are shown in FIG. 3(C).

FIG. 4 is a photographic representation showing that sodium selenate, but not selenomethionine, inhibits chronic activation of Akt in PTEN deficient PC-3 cells. (A) PC-3 cells were serum starved for 16 hours, then sodium selenate (Na₂SeO₄, 0.5 mM) added for the indicated periods and whole cell lysates (75 μg) loaded on 10% SDS-PAGE gels and membranes probed with phospho-specific Akt and pan-Akt antibodies. The results are shown in FIG. 4(A). (B) Identical lysates (75 μg) from (A) were probed with phospho-specific PDK1 antibodies and βIII tubulin antibody as a loading control. The results are shown in FIG. 4(B). (C) PC-3 cells were serum starved for 16 hours then, sodium selenate (Na₂SeO₄, 0.5 mM, for 10 minutes) of LY294002 (50 μM for 1 hour) alone or in combination were added, then cells lysed and whole cells lysates (75 μg) resolved on 10% SDS-PAGE gels and membranes probed with phospho-specific and pan Akt antibodies. The results are shown in FIG. 4(C). (D) PC-3 cells were serum starved for 16 hours then treated with either sodium selenate (Na₂SeO₄, 0.5 mM) or selenomethionine (SeMet, 0.5 mM) for the different time periods indicated then equal amounts of whole cell lysates (75 μg) resolved on 10% SDS-PAGE gels and membranes probed with phospho-specific and pan Akt antibodies. The results are shown in FIG. 4(D).

FIG. 5 is a graphical and photographic representation showing that sodium selenate, but not selenomethionine, induces nuclear translocation of Forkhead transcription factor and downregulation of PI3K cell survival pathway effector protein activity. (A) PC-3 cells were transfected with GFP-Forkhead expression construct (GFP-FKHR, 0.8 μg) with Lipofectamine 2000 reagent, then 24 hours later they were serum starved for 16 hours then treated with either sodium selenate (Na₂SeO₄, 0.5 mM), or selenomethionine (Se Met, 0.5 mM) for 3 hours, or LY294002 (10 μIM) for 1 hour. Cells were then fixed and nuclear and cytoplasmic GFP fluorescence status observed. DAPI stain indicates cell nuclei. The results are shown in FIG. 5(A). (B) FIG. 5(B) provides a graphical depiction of means +S.D. of results in (A). All experiments were performed in triplicate and at least 70 transfected cells were scored for each treatment. (C), (D) PC-3 cells were serum starved for 16 hours then treated with sodium selenate (Na₂SeO₄, 0.5 mM) for the indicated time periods and whole cell lysates (75 μg in (C) and 100 μg in (D) resolved on SDS-PAGE gels and membranes proved with the indicated antibodies. βIII tubulin served as a loading control in (C). Asynchronously growing PC-3 cells were treated with either sodium selenate (Na₂SeO₄) at the indicated concentrations with or without LY294002 (10 μM) for 72 hours. Cells were then processed for proliferative index using the MTT assay. Results depict the means +S.D. at least three independent experiments.

FIG. 6 is a graphical and photographic representation showing in vivo results of treatment of prostate tumor in mice with selenium in the form of selenate alone or in combination with taxol (paclitaxel). The treatments include: Cont (control group), selenium (sodium selenate) and taxol (paclitaxel). The control group was treated with the paclitaxel solubilization carrier cremophor and ethanol without paclitaxel. The Y-axis of the graph indicates prostate tumor weight (mg). Figures represent the means ± SD.

FIG. 7 is a photographic representation showing tissue sections of prostate tumors treated with taxol (paclitaxel) alone or taxol in combination with sodium selenate. Sodium selenate and paclitaxel synergize to reduce prostate tumor size. FIG. 7 shows images of tumor sections from paclitaxel alone treated animals (taxol) versus sodium selenate and paclitaxel combination treated animals (taxol+selenate). Tumor sections were stained with HE. Both images were taken at the same magnification and are directly comparable.

FIG. 8 is a graphical representation showing in vivo results of treatment of prostate tumors in mice with taxol (paclitaxel) (T) alone or in combination with sodium selenate (S+T). The Y-axis of the graph indicates prostate tumor volume (mm³). Figures represent the means + SD.

FIG. 9 is a graphical representation depicting the cell toxicity effects of 5 μM or 50 μM sodium selenate or sodium selenite measured by Trypan Blue Exclusion after 24 hours, 48 hours, 72 hours and 96 hours. Selenite and selenate Cell Toxicity are measured by Trypan Blue Exclusion. The results depict the means± SD of three independent experiments. The percentage of viable cells compared to total cell numbers in each sample are indicated on the y-axis and treatment of the x-axis.

FIG. 10 is a photographic representation showing the effect of taxol (paclitaxel) at 1 μg/mL or 10 μg/mL or sodium selenate 500 μM (equivalent to a dose of 19 mg/kg) or sodium selenite 500 μM (equivalent to a dose of 18 mg/kg) for the indicated times on PC-3 cells. Treated cell lysates were run on a SDS-PAGE gel and then blotted and probed with the indicated antibodies. Selenite is shown to induce DNA damage whilst selenate and paclitaxel do not. Whole cell lysates from treated PC-3 cells were transferred to PVDF membranes and probed with a phosphorylation specific Histone H2A.X antibody (P H2A.X) and then the membrane stripped and reprobed with a β-tubulin antibody as a loading control (Tubulin).

FIG. 11 is a graphical representation showing the effects of different selenium compounds on activation of Akt. Treatments: control (con); sodium selenate (ATE); Selenous acid (Sel acid); sodium selenite (ITE); selenium dioxide (SeO₂); selenium sulfide (SeS₂); methyl selenocysteine (MSC); and selenocysteine (SeC). Relative active Akt signal intensity correlated to total Akt protein levels is depicted on the y-axis. The graph indicates that only sodium selenate (ATE) inhibits activation of Akt, reducing levels of phosphorylated Akt below control (con) levels. In contrast, selenous acid (Sel acid), sodium selenite (ITE), selenium dioxide (SeO₂), selenium sulfide (SeS₂), methyl selenocysteine (MSC), selenocysteine (SeC) all induce activation of Akt above control (con) levels.

FIG. 12 is a photographic representation showing the effects of a high dose of sodium selenite and sodium selenate on Akt activation. Sodium selenite (ITE) 500 μM does not inhibit Akt activation in comparison to a similar dose of sodium selenate (ATE). Treated PC-3 cell lysates were run on a SDS-PAGE gels and blotted and probed with phosphorylation specific Akt antibody (ser 408) P-Akt antibody.

FIG. 13 is a photographic representation showing the results of the treatment of PC-3 cells with taxol (paclitaxel) at 1, 10, 100 ng/mL and 1 μg/mL, 10 μg/mL or sodium selenate (ATE) at 100 μM, 250 μM or 500 μM (equivalent to a dose of 4-19 mg/kg) or sodium selenite (ITE) at 100 μM, 250 μM or 500 μM (equivalent to a dose of 3.6-18 mg/kg) for 16 hours. The treated cell lysates were run on a SDS-PAGE gel and blotted and probed with specific antibodies directed to cleaved PARP protein and β-Tubulin (control). Taxol and selenate are shown to induce cleavage of the pro-apoptotic PARP protein whilst selenite does not. Selenite also induces marked degradation of cellular β-tubulin underlying its cellular toxicity.

FIG. 14 is a graphical and photographic representation showing the percentage inhibition of growth of parental LNCaP cells grown in the presence or absence of androgen after 3 days treatment with sodium selenate (50 μM). 5×10⁴ human prostate cancer androgen sensitive LNCaP cells were seeded in a 6-well dish and 8 hours later were treated with 50 μM sodium selenate, or no selenate (control). Cells were harvested at 72 hours following addition of the selenate and cell counts of viable cells as assessed by Trypan Blue staining were determined.

FIG. 15 is a graphical representation showing the percentage inhibition of growth of CSS LNCaP cell line grown in the presence or absence of androgen after 3 days treatment with sodium selenate (50 μM). 5×10⁴ human prostate cancer androgen independent CSS LNCaP cells were seeded in a 6-well dish and 8 hours later were treated with either 50 μM sodium selenate, or no selenate (control). Cells were harvested at 72 hours following addition of the sodium selenate and cell counts of viable cells as assessed by Trypan Blue staining were determined.

FIG. 16 is a graphical representation showing a time course of cell proliferation for parental LNCaP (normal serum, NS LNCaP) cells grown in the presence of androgen, following treatment with sodium selenate (5 μM) or LY294002 (10 μM). 1×10⁵ human prostate cancer androgen sensitive NS LNCaP cells were seeded in a 6-well plate and 8 hours later were treated with sodium selenate (5 μM), LY294002 (10 μM) or no treatment (control). Cells were harvested at 3 days, 5 days and 9 days following the addition of selenate or LY294002 and cell counts of viable cells as assessed by Trypan Blue staining were determined.

FIG. 17 is a graphical representation showing a time course of cell proliferation for parental NS LNCaP cells grown in the absence of androgen (in charcoal stripped serum, CSS) following treatment with sodium selenate (5 μM) or LY294002 (10 μM). 1×10⁵ human prostate cancer sensitive NS LNCaP cells were seeded in a 6-well dish and 8 hours later were treated with 5 μM sodium selenate, 10 μM LY294002 or no treatment (control). Cells were harvested at 3 days, 5 days and 9 days following the addition of selenate or LY294002 and cell counts of viable cells as assessed by Trypan Blue staining were determined.

FIG. 18 is a graphical representation showing a time course of cell proliferation for androgen independent LNCaP (CSS LNCap) cells grown in the presence of androgen (in normal serum, NS), following treatment with sodium selenate (5 μM) or LY294002 (10 μM). 1×10⁵ human prostate cancer androgen independent CSS LNCaP cells were seeded in a 6-well dish and 8 hours later were treated with 5 μIM sodium selenate, 10 μM LY294002 or no treatment (control). Cells were harvested at 3 days, 5 days and 9 days following the addition of sodium selenate or LY294002 and cell counts of viable cells as assessed by Trypan Blue staining were determined.

FIG. 19 is a graphical representation showing a time course of cell proliferation for androgen independent LNCaP (CSS LNCaP) cells grown in the absence of androgen (in charcoal stripped serum, CSS) following treatment with sodium selenate (5 μM) or LY294002 (10 μM). 1×10⁵ human prostate cancer androgen independent CSS LNCaP cells were seeded in a 6-well dish and 8 hours later were treated with either 5 μM sodium selenate or 10 μM LY294002 or no treatment (control). Cells were harvested at 3 days, 5 days and 9 days following addition of the sodium selenate or LY294002 and cell counts of viable cells as assessed by Trypan Blue staining were determined.

FIG. 20 is a graphical representation showing a time course of cell proliferation of Q293 cells grown in the presence of androgen (normal serum, NS) following treatment with sodium selenate (5 μM) or LY294002 (10 μM) or no treatment (control). 1×10⁵ human kidney epithelial Q293 cells were seeded in a 6-well dish and 8 hours later were treated with either 5 μM sodium selenate or 10 μM LY294002 or no treatment (control). Cells were harvested at 3 days, 5 days and 9 days following treatment with sodium selenate or LY294002 and cell counts of viable cells as assessed by Trypan Blue staining were determined.

FIG. 21 is a graphical representation showing a time course of cell proliferation of Q293 cells grown in the absence of androgen (in charcoal stripped serum, CSS) following treatment with sodium selenate (5 μM) or LY294002 (10 μM). 1×10⁵ human kidney epithelial Q293 cells were seeded in a 6-well dish and 8 hours later were treated with 5 μM sodium selenate or 10 μM LY294002 or no treatment (control). Cells were harvested at 3 days, 5 days and 9 days following addition of sodium selenate or LY294002 and cell counts of viable cells as assessed by Trypan Blue staining were determined.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 20%, or 10% to a reference quantity, level, value, dimension, size, or amount.

As used herein, the term “Akt signaling pathway activation-inhibiting amount” in the context of treating or preventing a cancer or inhibiting the growth of tumor cells is meant the administration of an amount or series of doses of selenate, which is effective in antagonizing the Akt signaling pathway, including preventing or reducing activation of Akt by preventing or reducing the expression of Akt or an upstream member of the pathway, or by reducing the level or functional activity of an expression product of the Akt gene or of or an upstream gene member of the pathway, or by preventing or reducing phosphorylation of Akt. The amount will vary depending on the health and physical condition of the individual to be treated, the taxonomic group of the individual to be treated, the formulation of the composition, the assessment of the medical situation, and other relevant factors. It is expected that the amount will fall within a relatively broad range that can be determined by routine trials. In specific embodiments, an Akt signaling pathway activation-inhibiting amount is a supranutritional amount of selenate.

By “androgen” is meant a hormone that encourages the development of male sexual characteristics. Non-limiting examples of androgens include testosterone, androstenedione, dihydroepiandrosterone and dihydrotestosterone.

The term “androgen-dependent cancer” or “androgen-dependent tumor cell” refers to a cancer or tumor cell that depends on an androgen for cell survival, growth and/or proliferation. Typically, an “androgen-dependent cancer” results from excessive accumulation of an androgen (e.g., testosterone or other androgenic hormone), increased sensitivity of androgen receptors to androgen, or an increase in androgen-stimulated transcription, and will generally benefit from a decrease in androgen stimulation.

The term “androgen-independent cancer” or “androgen-independent tumor cell” refers to a cancer or tumor cell which is insensitive to the presence or absence of androgens.

The phrases “cancer in which the Akt signaling pathway is activated” and “tumor cells in which the Akt signaling pathway is activated” refer to cancers and tumor cells in which the key cell survival pathway, the PTEN/PI3K/Akt pathway, is deregulated. Without wishing to be bound by any one theory or mode of operation, it is considered that activation of phosphoinositide 3-kinase PI3K, induced by diverse trophic signals, leads to the submembrane accumulation of the phosphorylated lipid products, phosphatidylinositol-3,4,5-triphosphate (PIP3) and phosphotidylinositol-3,4-bisphosphate (PIP2) (Vanhaesebroeck et al 2001, Annu. Rev. Biochem. 70:535; Cantley et al 2002, Science, 296:1655). The increased concentration of these phospholipids in the submembrane microenvironment, in turn, leads to the accumulation of the phosphoinositide-dependent kinases (PDK-1 and PDK-2), leading to activation of the serine/threonine kinase, Akt. The tumor suppressor protein PTEN, normally acts as an important negative regulator of PI3K by means of its action as a lipid phosphatase converting PIP3 back to PIP2. Inactivation of PTEN or loss of PTEN function results in chronic activation of the Akt signaling pathway. Tumor cells in which Akt is activated or in which PTEN is inactivated include various types of tumor cells, including carcinomas. For example, cancers in which Akt is activated or in which PTEN is inactivated include, but are not limited to oral squamous cell carcinoma, thyroid cancer, pituitary cancer, hepatocellular cancer including hepatocellular carcinoma, prostate cancer including prostate carcinoma, testicular cancer, fibrosarcoma, ovarian cancer including ovarian carcinoma, uterine cancer including endometrial cancer, pancreatic cancer including pancreatic carcinoma, stomach cancer, breast cancer, lung cancer, renal cell carcinoma, colon cancer, melanoma, acute leukemia and brain cancer (e.g: astrocytoma and glioblastoma). In some embodiments, the cancer is a hematopoietic neoplastic disorder, including diseases involving hyperplastic/neoplastic cells of hematopoietic origin, such as: lymphoma and a lymphocytic leukemia. Non-limiting examples of lymphomas include: T-cell lymphomas (including peripheral T-cell lymphomas, adult T-cell leukemia/lymphomas (ATL); cutaneous T-cell lymphomas (CTCLs); large granular lymphocytic leukemias (LGFs); B-cell lymphomas, Hodgkin's lymphoma and a non-Hodgkin's lymphoma. Illustrative examples of lymphocytic leukemias include: poorly differentiated acute leukemias, e.g., acute megakaryoblastic leukemia; myeloid disorders, including, but not limited to, acute promyeloid leukemia (APML), acute myelogenous leukemia (AML) and chronic myelogenous leukemia (CML); lymphoid malignancies, including, but not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

The phrases “cancer in which the Akt pathway is over-activated,” “cancer in which Akt is over-activated” and the like refer to a cancer in which not only Akt is overexpressed, but the kinase activity of Akt is positively enhanced by phosphorylation of the Thr 308 and Ser 473 residues. This is exemplified by the Akt isoform Akt3, where in primary breast cancer and prostate cancer cell lines, expression levels of Akt3 mRNA are some 2-4 fold higher than in normal cells but Akt3 kinase activity is elevated some 20-60 fold (Nakatani et al 1999, J. Biol. Chem. 274:21528). Akt is often over-activated in drug-resistant or refractory tumors or cancers. Examples of cancers in which Akt is over-activated or constitutively activated include, but are not limited to, androgen-independent prostate cancer and estrogen receptor-deficient breast cancer.

The term “carcinoma” as used herein refers to a form of cancer which develops in epithelial cells covering or lining organs such as the skin, the uterus, the lung, the breast or the prostate. Carcinomas may, but do not necessarily, directly invade nearby organs or may metastasize to distant sites such as liver, lymph nodes or bones.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “cytostatic agent” refers to a substance that can inhibit cell proliferation or cell division without necessarily killing the cell. Suitably, the cytostatic agent inhibits the proliferation of cancer cells.

The term “cytotoxic agent” or “cytotoxic therapy” as used herein refers to a substance or therapy that is harmful to cells and ultimately causes cell death. In some embodiments, the cytotoxic agent harms rapidly dividing cells such as cancer cells and causes cancer cell death, especially cancer cell death while not causing damage to or causing less damage to non-cancer cells. An example of a cytotoxic therapy is radiotherapy.

As used herein the terms “drug resistant” and “refractory” refer to a cancer or tumor cell which is unresponsive or partially unresponsive to treatments normally used to treat the cancer or kill the tumor cell.

The terms “hormone ablation” and “hormone ablation therapy” refer to the deprivation of hormones that may be required for the survival and growth of cancer cells. Hormone ablation may be achieved by surgical removal of hormone-producing organs such as testes or ovaries or may be achieved chemically with compounds that interfere with hormone biosynthesis or secretion, compounds that antagonize or block hormone receptors or in some way prevent a hormone exerting its biological effect. For example, the conversion of testosterone to the more active dihydrotestosterone may be blocked by a 5-alpha reductase inhibitor such as finasteride.

The term “hormone-dependent cancer” or “hormone-dependent tumor cell” refers to a cancer or tumor cell which depends on the presence of a hormone for survival, growth and/or proliferation. Hormone-dependent cancers include but are not limited to, prostate cancer, testicular cancer, breast cancer, ovarian cancer, uterine cancer, endometrial cancer, thyroid cancer and pituitary cancer.

As used herein, the term “hormone-dependent tumor cell growth-inhibiting amount” in the context of treating or preventing a cancer or inhibiting the growth of tumor cells is meant the administration of an amount or series of doses of selenate, which is effective in inhibiting the growth and/or proliferation of cancer or tumor cells or for causing tumor cell death. The amount will vary depending on the health and physical condition of the individual to be treated, the taxonomic group of the individual to be treated, the formulation of the composition, the assessment of the medical situation, and other relevant factors. It is expected that the amount will fall within a relatively broad range that can be determined by routine trials. In specific embodiments, a hormone-dependent tumor cell growth-inhibiting amount is a supranutritional amount of selenate.

As used herein the term “in combination with” refers to the treatment of cancer or exposure of a tumor cell to at least two agents such that their effects on the cancer or tumor cell occur, at least in part, over the same time period. Administration of at least two agents may occur simultaneously in a single composition, or each agent may be simultaneously or sequentially administered in separate compositions.

The phrase “inhibiting growth of tumor cells” is taken to mean that tumor cell growth is ceased or reduced and cell proliferation or cell division is ceased or reduced. This is also known as cytostasis. The growth of tumor cells can be measured in terms of weight or volume or cell number or cellular metabolic activity, i.e. MTT assay.

By “pharmaceutically acceptable carrier” it is meant a solid or liquid filler, diluent or encapsulating substance that may be safely used in topical, local or systemic administration.

The term “pharmaceutically acceptable salt” as used herein in relation to selenate refers to metal ion salts which are toxicologically safe for human and animal administration. For example, suitable metal ion salts of selenate include, but are not limited to, sodium, potassium, magnesium, calcium, iron, nickel and zinc salts. A preferred salt of selenate is the sodium salt, Na₂SeO₄.

The term “radiotherapy” as used herein refers to the treatment or exposure of a cancer or cancer cells such as tumor cells to high energy radiation. The effectiveness of radiotherapy may be enhanced by selenate or its pharmaceutically acceptable salt. Furthermore, radiotherapy may be further enhanced by administration of radiosensitizing agent. Illustrative examples of radiosensitizing agents include but are not limited to efaproxiral, etanidazole, fluosol, misonidazole, nimorazole, temoporfin and tirapazamine.

The terms “subject” or “individual” or “patient”, used interchangeably herein, refer to any subject, particularly a vertebrate subject and more particularly a mammalian subject, for whom prophylaxis or treatment is desired. Suitable vertebrate animals that fall within the scope of the invention include, but are not limited to, primates, avians, livestock animals (e.g., pigs, sheep, cows, horses, donkeys), laboratory test animals (e.g., rabbits, mice, rats, guinea pigs, hamsters), companion animals (e.g., cats and dogs) and captive wild animals (e.g., foxes, deer, dingoes). A preferred subject is a human in need of treatment or prophylaxis of a cancer in which the Akt signaling pathway is activated or of a hormone-dependent cancer, especially prostate cancer. However, it will be understood that the aforementioned terms do not imply that symptoms are present.

The term “supranutritional” as used herein, refers to an amount which is greater than the amount considered as a nutritional requirement. In adults, the recommended dietary allowance of selenium is 55 μg/day. Pregnant and lactating women have a recommended daily allowance of 60-70 μg/day. A supranutritional amount of selenium provides selenium to a subject at above the recommended daily allowance. For example, a supranutritional amount of selenium may be 0.15 mg/kg to 20.0 mg/kg, 0.1 mg/kg to 14 mg/kg, 0.1 mg/kg to 13 mg/kg, 0.1 mg/kg to 12 mg/kg, 0.1 mg/kg to 10 mg/kg, 0.1 mg/kg to 9 mg/kg, 0.1 mg/kg to 8 mg/kg, 0.1 mg/kg to 7 mg/kg, 0.1 mg/kg to 6 mg/kg, 0.15 mg/kg to 5 mg/kg, 0.15 mg/kg to 4 mg/kg, 0.15 mg/kg to 3 mg/kg, 0.15 mg/kg to 2 mg/kg, 0.15 mg/kg to 1 mg/kg, especially 0.1 mg/kg to 14 mg/kg and more especially 0.15 mg/kg to 5 mg/kg.

As used herein, the term “therapeutically effective amount” in the context of treating or preventing cancer or inhibiting the growth of tumor cells is meant the administration of an amount of selenate or a pharmaceutically acceptable salt thereof, either in a single dose or as part of a series of doses, that is effective for inhibiting the growth and/or proliferation of cancer or tumor cells or for causing cancer or tumor cell death. The effective amount will vary depending on the health and physical condition of the individual to be treated, the taxonomic group of the individual to be treated, and the formulation of the composition, the assessment of the medical situations and other relevant factors. It is expected that the amount will fall within a relatively broad range that can be determined through routine trials. In specific embodiments, a therapeutically effective amount is a supranutritional amount.

### 2. Methods for Inhibiting Tumor Cell Growth and Proliferation and for Treating Cancer

The present invention is predicated in part on the determination that selenate, as opposed to other forms of selenium such as selenite, is effective in inhibiting the growth or proliferation of tumor cells in which the Akt signaling pathway is activated. Accordingly, in one aspect, the present invention provides methods for inhibiting the growth or proliferation of tumor cells in which the Akt signaling pathway is activated, wherein the methods generally comprise exposing the tumor cells to an Akt signaling pathway activation-inhibiting amount of selenate or a pharmaceutically acceptable salt thereof. Suitably, the amount of selenate or its pharmaceutically acceptable salt is a supranutritional amount, which is generally from about 0.015 mg/kg to 20.0 mg/kg, usually from about 0.1 m/kg to 14 mg/kg and more usually from about 0.15 mg/kg to 5 mg/kg.

The present invention can be used effectively against various types of tumor cells and cancers, including carcinomas, illustrative examples, cancers in which Akt is activated or in which PTEN is inactivated include, but are not limited to oral squamous cell carcinoma, thyroid cancer, pituitary cancer, hepatocellular cancer including hepatocellular carcinoma, prostate cancer including prostate carcinoma, testicular cancer, fibrosarcoma, ovarian cancer including ovarian carcinoma, uterine cancer including endometrial cancer, pancreatic cancer including pancreatic carcinoma, stomach cancer, breast cancer, lung cancer, renal cell carcinoma, colon cancer, melanoma, acute leukemia and brain cancer (e.g. astrocytoma and glioblastoma). In some embodiments, the cancer is a hematopoietic neoplastic disorder, including diseases involving hyperplastic/neoplastic cells of hematopoietic origin, such as: lymphoma and a lymphocytic leukemia. Non-limiting examples of lymphomas include: T-cell lymphomas (including peripheral T-cell lymphomas, adult T-cell leukemia/lymphomas (ATL); cutaneous T-cell lymphomas (CTCLs); large granular lymphocytic leukemias (LGFs); B-cell lymphomas, Hodgkin's lymphoma and a non-Hodgkin's lymphoma. Illustrative examples of lymphocytic leukemias include: poorly differentiated acute leukemias, e.g., acute megakaryoblastic leukemia; myeloid disorders, including, but not limited to, acute promyeloid leukemia (APML), acute myelogenous leukemia (AML) and chronic myelogenous leukemia (CML); lymphoid malignancies, including, but not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). In specific embodiments, the tumor cells are prostate cancer or carcinoma cells. Thus, in a related aspect, the invention provides methods for treating a cancer in which Akt is activated, wherein the methods generally comprise administering to a subject in need thereof an Akt signaling pathway activation-inhibiting amount of selenate or a pharmaceutically acceptable salt thereof. In some embodiments, the amount of selenate or pharmaceutically acceptable salt thereof is a supranutritional amount as broadly defined above. In some embodiments, the cancer is prostate cancer, especially a drug resistant prostate cancer or an androgen-independent prostate cancer.

In some embodiments, the tumor cell or cancer is drug resistant. In some embodiments, the tumor cell or cancer has an overactive Akt pathway (e.g., androgen-independent prostate cancer and estrogen receptor-deficient breast cancer).

In certain embodiments, the selenate or its pharmaceutically acceptable salt is administered in combination with at least one cytostatic agent or cytotoxic agent. Non-limiting examples of cytostatic agents are selected from: (1) microtubule-stabilizing agents such as but not limited to taxanes, paclitaxel, docetaxel, epothilones and laulimalides; (2) kinase inhibitors, illustrative examples of which include Iressa®, Gleevec, Tarceva™, (Erlotinib HCl), BAY-43-9006, inhibitors of the split kinase domain receptor tyrosine kinase subgroup (e.g., PTK787/ZK 222584 and SU11248); (3) receptor kinase targeted antibodies, which include, but are not limited to, Trastuzumab (Herceptin®), Cetuximab (Erbitux®), Bevacizumab (Avastin™), Rituximab (ritusan®), Pertuzumab (Omnitarg™); (4) mTOR pathway inhibitors, illustrative examples of which include rapamycin and CCI-778; (5) Apo2L/Trail, anti-angiogenic agents such as but not limited to endostatin, combrestatin, angiostatin, thrombospondin and vascular endothelial growth inhibitor (VEGI); (6) antineoplastic immunotherapy vaccines, representative examples of which include activated T-cells, non-specific immune boosting agents (i.e., interferons, interleukins); (7) antibiotic cytotoxic agents such as but not limited to doxorubicin, bleomycin, dactinomycin, daunorubicin, epirubicin, mitomycin and mitozantrone; (8) alkylating agents, illustrative examples of which include Melphalan, Carmustine, Lomustine, Cyclophosphamide, Ifosfamide, Chlorambucil, Fotemustine, Busulfan, Temozolomide and Thiotepa; (9) hormonal antineoplastic agents, non-limiting examples of which include Nilutamide, Cyproterone acetate, Anastrozole, Exemestane, Tamoxifen, Raloxifene, Bicalutamide, Aminoglutethimide, Leuprorelin acetate, Toremifene citrate, Letrozole, Flutamide, Megestrol acetate and Goserelin acetate; (10) gonadal hormones such as but not limited to Cyproterone acetate and Medoxyprogesterone acetate; (11) antimetabolites, illustrative examples of which include Cytarabine, Fluorouracil, Gemcitabine, Topotecan, Hydroxyurea, Thioguanine, Methotrexate, Colaspase, Raltitrexed and Capicitabine; (12) anabolic agents, such as but not limited to, Nandrolone; (13) adrenal steroid hormones, illustrative examples of which include Methylprednisolone acetate, Dexamethasone, Hydrocortisone, Prednisolone and Prednisone; (14) neoplastic agents such as but not limited to Irinotecan, Carboplatin, Cisplatin, Oxaliplatin, Etoposide and Dacarbazine; and (15) topoisomerase inhibitors, illustrative examples of which include topotecan and irinotecan.

In some embodiments, the cytostatic agent is a nucleic acid molecule, suitably an antisense or siRNA recombinant nucleic acid molecule. In other embodiments, the cytostatic agent is a peptide or polypeptide. In still other embodiments, the cytostatic agent is small molecule. The cytostatic agent may be a cytotoxic agent that is suitably modified to enhance uptake or delivery of the agent. Non-limiting examples of such modified cytotoxic agents include, but are not limited to, pegylated or albumin-labelled cytotoxic drugs.

In specific embodiments, the cytostatic agent is a microtubule stabilizing agent, especially a taxane and preferably paclitaxel.

In some embodiments, the cytotoxic agent is selected from the anthracyclines such as idarubicin, doxorubicin, epirubicin, daunorubicin and mitozantrone, CMF agents such as cyclophosphamide, methotrexate and 5-fluorouracil or other cytotoxic agents such as cisplatin, carboplatin, bleomycin, topotecan, irinotecan, melphalan, chlorambucil, vincristine, vinblastine and mitomycin-C.

The present invention also discloses the discovery that selenate and its pharmaceutically acceptable salts have an inhibitory effect on hormone-dependent tumor cell growth when used in combination with a hormone ablation therapy and optionally a cytostatic agent or cytotoxic agent. Accordingly, another aspect of the present invention provides methods for treating a hormone-dependent cancer in a subject, wherein the methods generally comprise administering a therapeutically effective amount of selenate or a pharmaceutically acceptable salt thereof in combination with a hormone ablation therapy. Suitably, the amount of selenate or a pharmaceutically acceptable salt thereof is a supranutritional amount of selenate, as broadly defined above. In some embodiments, the hormone-dependent cancer is selected from prostate cancer, testicular cancer, breast cancer, ovarian cancer, endometrial cancer, uterine cancer, thyroid cancer or pituitary cancer, especially prostate cancer or breast cancer.

The hormone ablation therapy may be any therapy that deprives the cancer or tumor cells of hormones required for cancer or tumor cell survival, growth and/or proliferation. Hormone ablation therapy may be achieved surgically by removal of hormone-producing organs such as testes or ovaries. Alternatively, the hormone ablation therapy may be achieved chemically with compounds that interfere with hormone biosynthesis or secretion, compounds that antagonize or block hormone receptors or in some way prevent the hormone exerting its biological effect. Illustrative agents for chemical hormone ablation therapy include GnRH agonists or antagonists such as Cetrorelix, agents that interfere with the androgen receptor including non-steroidal agents such as Bicalutamide and steroidal agents such as Cyproterone, and agents that interfere with steroid biosynthesis such as Ketoconazole. Chemical agents suitable for use in combination with selenate or its pharmaceutically acceptable salts as hormone ablation therapy for prostate cancer include, but are not limited to, non-steroidal anti-androgens such as Nilutamide, Bicalutamide and flutamide; GnRH agonists such as Goserelin acetate, leuprorelin and triptorelin; 5-alpha reductase inhibitors such as finasteride; and cyproterone acetate. Chemical agents suitable for use in combination with selenate or its pharmaceutically acceptable salts as hormone ablation therapy in breast cancer include but are not limited to aromatase inhibitors such as Anastrozole; Exemestane, Tamoxifen, Aminoglutethimide, Toremifene citrate, Letrozole, Megestrol acetate and Goserelin acetate. Chemical agents suitable for use in combination with selenate or its pharmaceutically accdeptable salts as hormone ablation therapy in ovarian and uterine cancers, including endometrial cancer, include but are not limited to, progestins such as Megestrol acetate, levonorgestrol and norgestrol.

In some embodiments, the method of treating a hormone-dependent cancer further comprises administering a cytostatic agent such as those defined above or a cytotoxic agent. A preferred cytostatic agent is a microtubule-stabilizing agent, especially a taxane, and more especially paclitaxel.

Certain embodiments of the present invention are directed to methods for treating cancer in a subject, which methods generally comprise administering to the subject a therapeutically effective amount of selenate or a pharmaceutically acceptable salt thereof. To practice these methods, the person managing the subject can determine the effective dosage form of selenate or its pharmaceutically acceptable salts for the particular condition and circumstances of the subject. A therapeutically effective amount of selenate is one that is effective for the treatment or prevention of the cancer, including the prevention of incurring a symptom (e.g., proliferation of cancer cells), holding in check such symptoms, and/or treating existing symptoms associated with the cancer (e.g., pain, fluid build-up, urinary retention, nausea, indigestion, gas, appetite, changes in bowel habits and weight loss). In some embodiments, the therapeutically effective amount is a supranutritional amount of selenate or its pharmaceutically acceptable salt. In specific embodiments, the selenate is suitably in the form of the salt, sodium selenate (Na₂SeO₄).

Modes of administration, amounts of selenate administered, and selenate formulations, for use in the methods of the present invention, are discussed below. Whether the cancer has been treated is determined by measuring one or more diagnostic parameters indicative of the course of the disease, compared to a suitable control. In the case of a human subject, a “suitable control” may be the individual before treatment, or may be a human (e.g., an age-matched or similar control) treated with a placebo. In accordance with the present invention, the treatment of cancer includes and encompasses without limitation: (i) preventing cancer in a subject who may be predisposed to the cancer but has not yet been diagnosed with the cancer and, accordingly, the treatment constitutes prophylactic treatment for the cancer; (ii) inhibiting tumorigenesis, i.e., arresting the development of cancer; or (iii) relieving symptoms resulting from the cancer.

The methods of the present invention are suitable for treating an individual who has been diagnosed with a cancer, who is suspected of having a cancer, who is known to be susceptible and who is considered likely to develop a cancer, or who is considered likely to develop a recurrence of a previously treated cancer.

In specific embodiments of the above methods, the cancer is prostate cancer, especially a drug-resistant or androgen-independent prostate cancer and the treatment optionally further comprises administration of a cytostatic agent such as those defined above (e.g., a microtubule stabilizing agent such as paclitaxel) or a cytotoxic agent.

In other embodiments, the prostate cancer is an androgen-sensitive prostate cancer and the treatment is optionally administered in combination with a hormone ablation therapy and/or a cytostatic agent as defined above or a cytotoxic agent or radiotherapy optionally together with a radiosensitizing agent. Suitably, the hormone ablation therapy is selected from surgical castration, finesteride, Nilutamide, Cyproterone acetate, Bicolutamide, Leuprorelin acetate, Flutamide and Goserelin acetate. Preferably the cytostatic agent is a microtubule stabilizing agent, especially a taxane, more especially paclitaxel.

Exemplary subjects for treatment with the methods of the invention are vertebrates, especially mammals. In certain embodiments, the subject is selected from the group consisting of humans, sheep, cattle, horses, bovine, pigs, dogs and cats. A preferred subject is a human.

The selenate or pharmaceutically acceptable salt may be formulated by following any number of techniques known in the art of anticancer drug delivery. Selenate or its pharmaceutically acceptable salts may of course be administered by a number of means keeping in mind that all formulations are not suitable for every route of administration. Selenate or its pharmaceutically acceptable salts can be administered in solid or liquid form. The application may be oral, rectal, nasal, topical (including buccal and sublingual), or by inhalation. Selenate or its pharmaceutically acceptable salts may be administered together with conventional pharmaceutical acceptable adjuvant, carriers and/or diluents.

The solid forms of application comprise tablets, capsules, powders, pills, pastilles, suppositories and granular forms of administration. They may also include carriers or additives, such as flavors, dyes, diluents, softeners, binders, preservatives, lasting agents and/or enclosing materials. Liquid forms of administration include solutions, suspensions and emulsions. These may also be offered together with the above-mentioned additives.

Solutions and suspensions of selenate or pharmaceutically acceptable salt thereof, assuming a suitable viscosity for ease of use, may be injected. Suspensions too viscous for injection may be implanted using devices designed for such purposes, if necessary. Sustained release forms are generally administered via parenteral or enteric means. Parenteral administration is another route of administration of the selenate or a pharmaceutically acceptable salt thereof used to practice the invention. “Parenteral” includes formulations suitable for injection and for nasal, vaginal, rectal, and buccal administration.

The administration of selenate or its pharmaceutically acceptable salts may involve an oral prolonged dose formulation. Oral dose formulations are preferably administered once daily to three times daily in the form of a sustained release capsule or tablet, or alternatively as an aqueous based solution. Selenate or its pharmaceutically acceptable salt may be administered intravenously either daily, continuously, once a week or three times a week.

The administration of selenate or its pharmaceutically acceptable salts may include daily administration, preferably once daily in the form of a sustained release capsule or tablet, or once daily as an aqueous solution.

Combinations of selenate or its pharmaceutically acceptable salt and at least one cytostatic agent or a cytotoxic agent may be administered in solid or liquid form in a single formulation or composition or in separate formulations or compositions. In some embodiments, the selenate or its pharmaceutically acceptable salt and the cytostatic agent(s) or cytotoxic agent(s) are administered orally as a single tablet or capsule or separate tablets or capsules. In other embodiments, the selenate or its pharmaceutically acceptable salt and the cytostatic agent(s) or cytotoxic agent(s), are administered intravenously in a single composition or separate compositions.

The methods of the present invention may be employed in combination with other known treatments for cancer, for instance but not limited to, surgery, chemotherapy and radiotherapy. In some embodiments, the selenate or its pharmaceutically acceptable salt is used in combination with radiotherapies, such as but not limited to, conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine. In other embodiments, selenate or its pharmaceutically acceptable salt is used in combination with a tumorectomy.

The present invention also provides pharmaceutical compositions for treating or preventing cancer, generally comprising a supranutritional amount, suitably from about 0.5 mg to about 1.0 g of selenate or a pharmaceutically acceptable salt thereof and a pharmaceutically acceptable carrier. In some embodiments, the selenate or its pharmaceutically acceptable salt is in an amount of about 5.0 mg to about 700 mg. In illustrative examples, the selenate or its pharmaceutically acceptable salt is an amount of about 7.5 mg to 250 mg, especially 50 mg to 200 mg, for example 100 to 150 mg for a single daily dose. The pharmaceutical composition may be for treating a cancer in which Akt is activated or over-activated or a hormone-dependent cancer. In some embodiments, the pharmaceutical compositions are useful for treating prostate cancer, especially a drug-resistant or androgen-independent prostate cancer.

In some embodiments, the pharmaceutical compositions further comprise at least one cytostatic agent or cytotoxic agent. In other embodiments, the pharmaceutical compositions further comprise a chemical hormone ablation agent. In still other embodiments, the pharmaceutical compositions further comprise at least one cytostatic agent and/or a cytotoxic agent and a chemical hormone ablation agent. In still other embodiments, the pharmaceutical compositions may further comprise a radiosensitizing agent for use with radiotherapy.

The pharmaceutical composition of the present invention may include any additional components that are non-immunogenic and biocompatible with selenate, as well as capable of bioabsorption, biodegradation, elimination as an intact molecule. The formulation may be supplied in a ready-to-use form or may be supplied as a sterile powder or liquid requiring vehicle addition prior to administration. If sterility is desired, the formulation may be made under sterile conditions, the individual components of the mixture may be sterile, or the formulation may be sterile filtered prior to use. Such a solution can also contain appropriate pharmaceutically acceptable carriers, such as but not limited to buffers, salts, excipients, preservatives, etc.

In some embodiments, sustained release oral formulations are used for administering selenate or its pharmaceutically acceptable salt in the methods of the invention. These formulations generally comprise selenate or its pharmaceutically acceptable salt having decreased solubility in order to delay absorption into the bloodstream. In addition, these formulations may include other components, agents, carriers, etc., which may also serve to delay absorption of the selenate or its pharmaceutically acceptable salt. Microencapsulation, polymeric entrapment systems, and osmotic pumps, which may or may not be bioerodible, may also be used to allow delayed or controlled diffusion of the selenate or a pharmaceutically acceptable salt thereof from a capsule or matrix.

The selenate or its pharmaceutically acceptable salts can be used solus or as part of another agent. Accordingly, the present invention also contemplates an agent that comprises selenate or a pharmaceutically acceptable salt thereof for the treatment of a cancer in which Akt is activated, or for the treatment of a hormone-dependent cancer in which the agent is formulated for administration in combination with hormone ablation therapy.

In order that the nature of the present invention may be more clearly understood and put into practical effect, particular preferred embodiments thereof will now be described with reference to the following non-limited examples.

### EXAMPLES

### Example 1

### SUPPRESSION OF PROSTATE TUMOR PROGRESSION WITH SODIUM SELENATE BY INHIBITING ACTIVATION OF PROTEIN KINASE B/AKT

### Material and Methods

### Cell Culture

Cell culture experiments involved a PC-3 cell line which was obtained from the American Type Culture Collection (Manassas, Va., USA). The cells were routinely cultured in RPMI 1641 (Invitrogen) supplemented with 10% foetal calf serum and 1% antibiotic/antimycotic mixture (Invitrogen). Cells were maintained at 37° C. in 5% CO₂. Sodium selenate and selenomethionine (Sigma) were made up as 10 mM stock solutions in distilled water and filter sterilized before dilution in media for in vitro experiments.

### Animal Experimentation

Animal experimentation involved the use of BALB/c nude mice. On day one of the experiment 6 week old BALB/c nude mice were anaesthetized with an intraperitoneal injection of ketamine 100 mg/kg and xylazine 20 mg/kg. Under magnification 1×10⁶ PC-3 cells with viability greater than 95%, were injected into the dorsolateral prostate, essentially as described in Corcoran, N. M., Najdovska, M., and Costello, A. J. (2004), J. Urol., 171:907-910.

On day 3 of the experiment all mice were switched to the D19101 minimal selenium diet with an assayed selenium content of 0.07 ppm (Research Diets Inc., New Brunswick, N.J., USA). Mice were then randomly assigned to receive either 5 ppm of selenium as sodium selenate in the drinking water or unsupplemented water. After 5 weeks of supplementation mice were culled and tumor-containing prostate glands were then weighed and measured with a Vernier calliper. The retroperitonium was then explored under magnification cephadally to the level of the renal veins and lymph nodes measuring greater than 0.5 mm identified.

The degree of apoptosis in tissue samples was determined using an in situ TUNEL Cell Death Detection Kit (Roche Applied Science) according to the manufacture's instructions. The number of brown staining cells at high power (×400) were counted in 10 randomly chosen fields in areas where necrosis was absent on H&E staining of adjacent sections.

The rate of tumor cell proliferation was determined by in vivo BrdU incorporation. Two hours prior to culling, the mice were injected with 50 mg/kg BrdU (Sigma) intraperitoneally. 5 μm sections were cut, fixed in absolute methanol at 4° C. for 10 minutes. Sections were rehydrated in PBS and incubated in 2 N HCl for 1 hour at 37° C. to denature the DNA. The acid was neutralized by immersing the slides in 0.1 M borate buffer (pH 8.5). Following 3×PBS washes the samples were incubated with a monoclonal antibody to bromodeoxyuridine (BrdU) (Roche Applied Science) at a concentration of 5 μg/mL diluted in 0.1% BSA in PBS for 1 hour at room temperature, then overnight at 4° C. Samples were washed 3× times in PBS and incubated for 1 hour with a biotinylated link immunoglobulin mixture (DAKO Corporation). After 3 washes in PBS, samples were incubated with streptavidin-horseradish peroxidase (BD Biosciences) and immunostaining revealed with DAB (Enhanced Liquid Substrate System, Sigma). 10 fields at high power (×400) were chosen at random and the number of brown staining cells counted.

### PC-3 Growth Curve

Analysis of PC-3 growth curves were achieved by allowing 2×10⁵ PC-3 cells to attach overnight. After 16 hours the medium was changed to include sodium selenate in the presence of serum at the indicated concentrations and allowed to grow until the specified time points. Supernatants and cells were then harvested, combined and viable cells assessed by Trypan Blue exclusion assay. Experiments were performed in triplicate.

### BrdU Incorporation Assays and Immunofluorescence

BrdU Incorporation assays and immunofluorescence were carried out by plating 2.5×10³ PC-3 cells and allowing them to attach overnight. After 16 hours the media was changed to include sodium selenate at the indicated concentrations in the presence of serum. 36 hours later the sodium selenate-containing medium was refreshed and 1 μL/mL of Cell Proliferation Labeling Reagent (BrdU/FrdU, Amersham Biosciences) added. Cells were incubated for a further 16 hours, then washed 3× in PBS and fixed in 4% PFA for 10 minutes at room temperature.

Mouse monoclonal anti-BrdU and antibodies were used as the primary antibodies and anti-mouse 488 Alexa, as the secondary antibodies. The percentage of cells incorporating BrdU was determined by counting the number of green staining nuclei per number of DAPI positive cells. 100 DAPI positive cells were counted per coverslip and the experiment performed in triplicate.

### Determination of Level of Cell Cycle Block

The determination of level of cell cycle block involved plating 5×10⁵ PC-3 cells that were synchronized by serum starvation for 48 hours before sodium selenate was added in fresh serum containing medium at the indicated concentrations. Cells were allowed to grow until the indicated time points, then harvested, washed in PBS and fixed in ice cold 70% ethanol for 15 minutes. Cells were washed with PBS and resuspended in PBS containing 40 μg/mL of propidum iodide (Sigma) and 100 μg/L of RNase. DNA histograms were generated for each reading and the proportion of cells present in the G1 and G2/M peaks determined. Results were obtained for three independent experiments.

### MTT Growth Assay

MTT Growth Assays involved plating 1×10³ PC-3 cells per well in a 96 well plate and allowing the cells to attach overnight. At 16 hours the medium was replaced with fresh media containing the indicated concentration of sodium selenate or selenomethionine. For the experiment determining the additional effect of PI3K inhibition on sodium selenate growth inhibition, LY294002 (Promega, Madison, Wis., USA) was added at a concentration of 50 μM with the sodium selenate. The controls for this experiment received an equal volume of LY294002 diluent (DMSO). MTT growth assays were then performed according to the manufacturer's protocol (Sigma).

### Antibodies and Immunoblotting

The following antibodies were obtained from Cell Signaling Technology (Beverly, Mass., USA) unless otherwise stated: anti-p27^{KIP1} (bd Pharmingen), anti-p21^{CIP1} , ANTI-CYCLIN d1, ANTI-CYCLIN d3, ANTI-CDK4, ANTI-CDK6, ANTI-PHOSPHO rb (Ser807/811), anti-RB, anti-phospho Akt (Ser473), anti-phospho Akt (Thre308), anti-Akt, anti-phospho PDK1 (Ser241), anti-phospho PDK1 (Tyr373/376), anti-phospho GSK-3β, anti-phospho mTOR (Ser2448), anti-mTOR, anti-βIII tubulin (Promega).

To determine the effect of sodium selenate on cell cycle regulatory protesin, 5×10⁵ PC-3 cells were treated with 0.5 mM sodium selenate for the indicated times. To determine the effect of selenate on phosphorylation of the retinoblastoma protein, 2×10⁵ cells were allowed to attach overnight, then serum starved for 24 hours before being treated with 0.5 mM sodium selenate for the indicated time. To determine the effect of selenate on the phosphorylation of proteins involved in the PI3K/Akt signaling cascade, 5×10⁵ PC-3 cells were allowed to attach for 10 hours. Cells were serum starved for 16 hours and then treated with 0.5 mM sodium selenate for the indicated times. Cells were lysed in ELB (250 mM NaCl, 50 mM HEPES pH 7.0, 5 mM EDTA, 0.5 mM DTT, 0.2% TX100, 20 mM sodium fluoride, 2 mM sodium pervanadate, Complete Protease Inhibitor Cocktail (Roche)). Lysates were centrifuged for 15 minutes at 4° C. Protein concentration was determined using the BCA system (Sigma) and equal amounts of protein were loaded into each lane of a SDS-polyacrylamide gel. The proteins were transferred onto PVDF (Millipore) membranes and detected with anti-mouse or anti-rabbit secondary antibodies couples to horseradish peroxidase (HRP) and chemiluminescence using the SuperSignal West Dura (Pierce). Membranes were stripped using the Restore Western Blot Stripping Buffer (Pierce).

### Cell Transfection and Immunofluorescence

The day before transfection, 5×10⁵ PC-3 cells were seeded in 24-well dishes and transiently transfected with 0.8 μg of pcDNA3-GFP-FKHRwt construct (kind gift Dr Bill Sellers, Dana-Farber Institute, Boston) using Lipofectamine 2000 (Invitrogen). Cells were permeabilized in 0.2% Triton X-100 and the nuclear contents stained with DAPI. Coverslips were mounted with fluorescent mounting medium (DAKO). The cellular distribution of green fluorescence was determined in dual fluorescent cells using a Leica epifluorescent microscope.

### Statistical Analysis

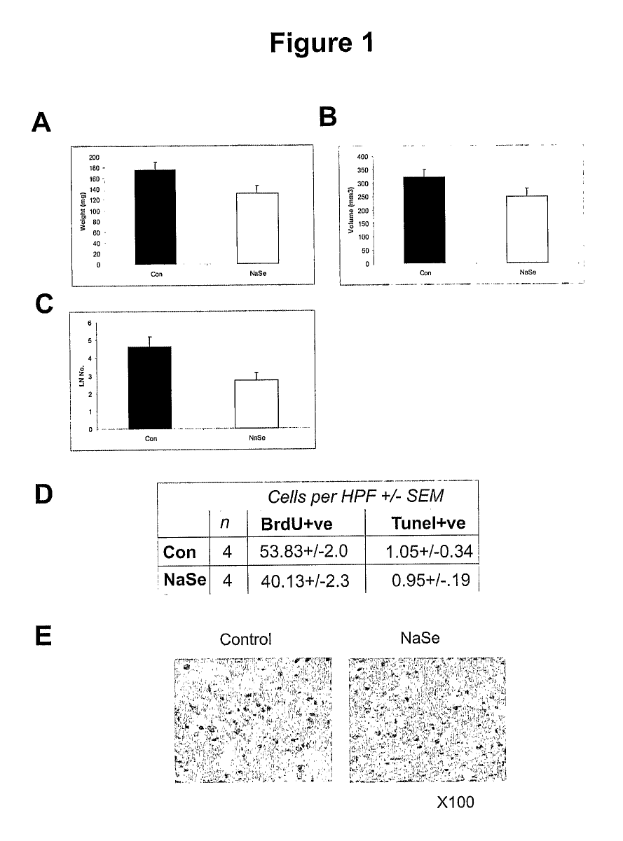

Date are presented as mean + SE unless otherwise indicated. Differences between groups (FIG. 1 A-D) were analyzed using Student's test with significance assumed at p<0.05. All statistical analysis was performed using SPSS 9.05 for Windows (SPSS, Chicago, Ill.) software.

### Results

The results indicated that sodium selenate inhibits proliferation of prostate carcinoma cells in an orthotopic mouse model. A comparison of sodium selenate treated animals with control group were made as follows: The mean tumor containing prostate weights were compared as shown in FIG. 1A and the mean tumor volumes were compared as shown in FIG. 1B. The mean number of lymph node metastases was also assessed. Sodium selenate was shown to significantly inhibit primary tumor growth compared with controls assessed by the prostate weight index, whilst tumor volume differences between the two groups just failed to reach significance. The number of retroperitoneal lymph nodes was significantly decreased in the sodium selenate group compared with controls (FIG. 1C).

The inhibition of tumor growth caused by sodium selenate may have occurred due to an inhibitory effect on tumor cell proliferation or through an increase in tumor cell apoptosis. To distinguish between these two mechanisms, the incorporation of the nucleotide analog, BrdU in proliferating cells and for apoptotic markers with the Tunel assay on control and sodium selenate treated prostates was analyzed. Sodium selenate significantly impaired BrdU incorporation in prostate tumors versus controls (FIGS. 1D, E), but apoptosis between the two groups was not significantly different (FIG. 1D). These results indicated that sodium selenate acts to impede tumor cell proliferation rather than enhance tumor cell death.

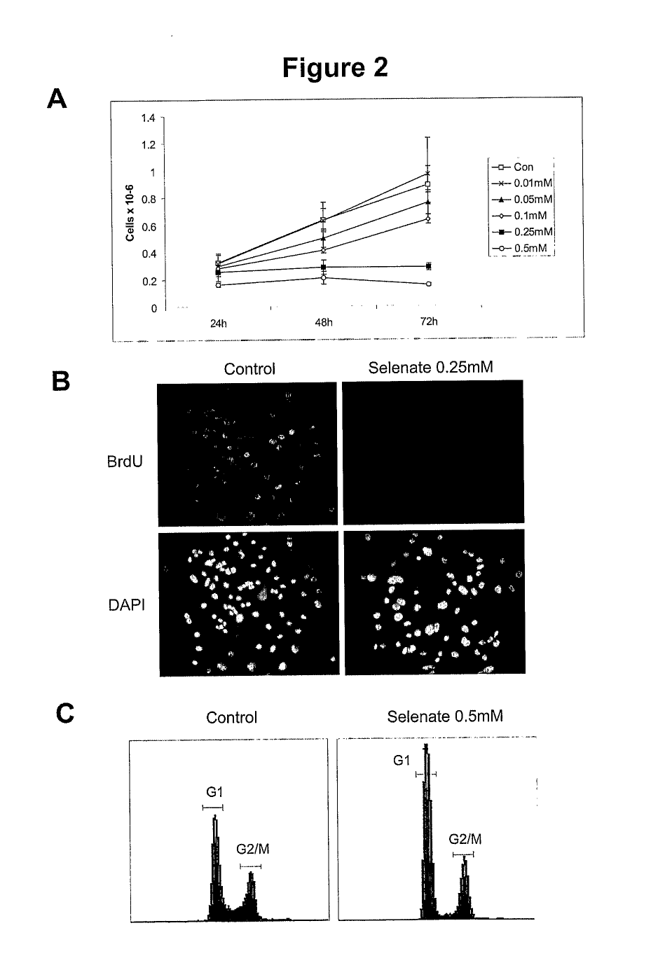

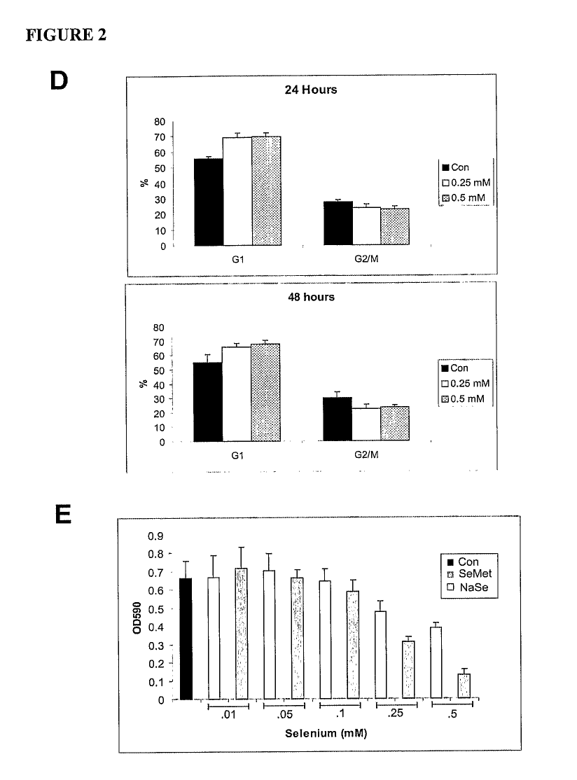

The results also indicated that sodium selenate arrests prostate carcinoma cells in G1-phase of the Cell Cycle. To elucidate whether the inhibitory effect of sodium selenate on tumor progression observed in vivo, would lead to an inhibition of tumor cell growth in vitro, human prostate carcinoma PC-3 cells were seeded and cultured for 16 hours. Cells were then incubated in the presence, or absence, of increasing concentrations of sodium selenate. Total cell numbers were counted 24, 48 and 72 hours after the incubation. As indicated in FIG. 2A, the number of PC-3 cells cultured in the absence of sodium selenate increased uniformly over the 72 h time period. In contrast, PC-3 cell numbers significantly declined in a dose-dependent manner in the presence of sodium selenate. Between doses of 0.01 and 0.1 mM, the rate of increase in cell number was impeded whilst at the higher doses of 0.25 and 0.5 mM, all increases in cell number were blocked. These results suggested that sodium selenate interferes with cell cycle progression, PC-3 cells were seeded essentially as described above, in concentrations of sodium selenate ranging from 0.05 to 0.25 mM, for 54 hours. Incorporation of the fluorescent nucleotide analogs BrdU/FrdU in cells progressing through G1/S phase was then determined by Immunofluorescence microscopy. A typical image of cells exposed to 0.25 mM sodium selenate for 54 hours is shown in FIG. 2B. Exposure to this dose of sodium selenate severely impeded G1/S progression of PC-3 cells as assess by BrdU/FrdU positive nuclei.

The cell cycle distribution in the presence, or absence of increasing concentrations of sodium selenate by flow cytometry was also analyzed. A typical histogram of the cell cycle is shown in FIG. 2C and mean percentage values in FIG. 2D. Treatment of PC-3 cells with 0.25 and 0.5 mM sodium selenate for 24 hours increased the percentage of the cell population in G1 from 55% in the control group to a range of 69-70% in the sodium selenate treated samples (FIG. 2D). Conversely, incubation in the presence of increasing concentrations of sodium selenate decreased the percentage of cells to 19% and 24% from the control of 28% at G2/M phase. Treatment with sodium selenate over a 48 hours time period increased the percentage of the cell population in G1 in a dose- and time-dependent manner, whilst the drug treatment decreased the percentage of PC-3 cells at G2/M phase.

The cancer chemopreventive effect of selenium cannot be fully explained by the presence of the selenium trace element, and it is becoming increasingly clear that the selenium compound itself is important in this activity (Corcoran et al 2004, supra; Kim et al 2003, supra). The effects of inorganic sodium selenate with organic selenomethionine on PC-3 cell proliferation were compared using the MTT assay as a measure of proliferative index. As shown in FIG. 2E, sodium selenate at doses of 0.25 and 0.5 mM markedly reduced PC-3 cell proliferation at the 72 hour period whilst selenomethionine was less effective in reducing cell proliferation at the same doses. A slight, but reproducible boost in cell number at the lowest dose of sodium selenate, 0.01 mM, was observed (FIG. 2E). Overall, these date indicate that sodium selenate inhibits cell proliferation by impeding cell cycle entry from G1 to S phase.

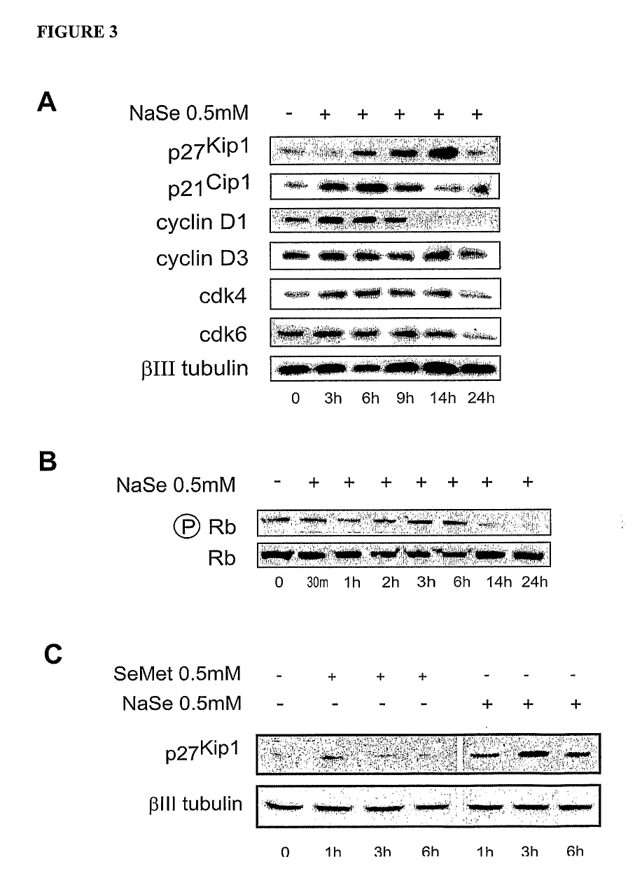

The results also indicated that sodium selenate, but not selenomethionine, induces up-regulation of cell cycle inhibitory proteins. The ordered progression of proliferating cells through GI is regulated principally by the sequential activation of the cyclin D/cdk4/cdk6 kinase complex which regulates the phosphorylation of Rb and the release of the E2F transcription factor. To determine the effect of sodium selenate treatment upon key cell cycle regulatory proteins, PC-3 cells were treated essentially as already described with 0.5 mM sodium selenate for different time periods out to 24 hours. Total cellular proteins were then resolved and analyzed by immunoblot assays for the presence of cyclin D1, D3cdk4 and cdk6. The expression levels of cyclin D1 dropped significantly from the 14 hour time period, whilst cdk4 levels peaked at 6 hours and declined in time dependent manner to 24 hours (FIG. 3A). CDK inhibitors such as p27^{KIP1} and p21^{CPI1/WAF1}, are important negative regulators of cell cycle progression. These molecules block CDK kinase activity by binding to cyclin D/CDK complex at G1 phase, preventing the phosphorylation of members of the Rb gene family and transition from G1 to S phase. To determine the effect of sodium selenate on these cell cycle inhibitors, the expression levels of p27^{KIP1} and p21^{CIP1} in the same PC-3 cell lysates were analyzed as described above. Treatment with sodium selenate increased p27^{KIP1} levels in the time-dependent manner till 24 hours when levels declines, whilst there was a marked increase in p21^{CIP1} levels, peaking at 6 hours and declining thereafter (FIG. 3A). A marked decline in phosphorylation of Rb (ser807/811) in a time-dependent manner in PC-3 cells treated with sodium selenate under the same conditions was also observed (FIG. 3B). The effects of inorganic versus organic selenium compounds on the levels of cell cycle inhibitor protein p27^{KIP1} were compared in Pc-3 cells. As shown in FIG. 3C, selenomethionine had little effect on p27^{KIP1} levels over the time period analyzed while sodium selenate, markedly boosted p27^{KIP1} total protein levels as compared to the control (0 hours) and selenomethionine levels (FIG. 3C). These data indicate the decline in cyclin D1 levels and phosphorylation of Rb as well as the concomitant increase in cell cycle inhibitory proteins p27^{KIP1} and p21^{CIP1/WAF1} that likely play an important role in regulating the G1 arrest induced by the inorganic selenium compound, and that organic selenium is unable to induce these effects.

The present results also indicate that sodium selenate, but not selenomethionine, potently inhibits protein kinase B/Akt activation. Loss of regulation of the PI3K cell survival pathway through loss of the tumor suppressor protein PTEN, is a common hallmark of human neoplasia (Vivanco, I. and Sawyers, C. L. 2002, supra), particularly prostate tumors (Visakorpi, 1999, Ann. Chir. Gynaecol., 88:11-16; Li et al 1997, Science, 275: 1943-1947). The PI3K pathway, through its effector kinase Akt, has an important role in regulating cellular proliferation, by preventing degradation of cyclin D1, and negatively influencing the expression of the cell cycle inhibitory proteins p27^{KIP1} and p21^{WAF1/CIP1} (Graff et al 2002, J. Biol. Chem., 275:24500-24505). PC-3 cells have lost expression of PTEN and have a constitutively activated PI3K pathway activity (Chakraborty et al 2001, Cancer Res., 61:7255-7263. Beresford et al 2001, J. Interferon Cytokine Res., 21:313-322), particularly the kinase activity of Akt.

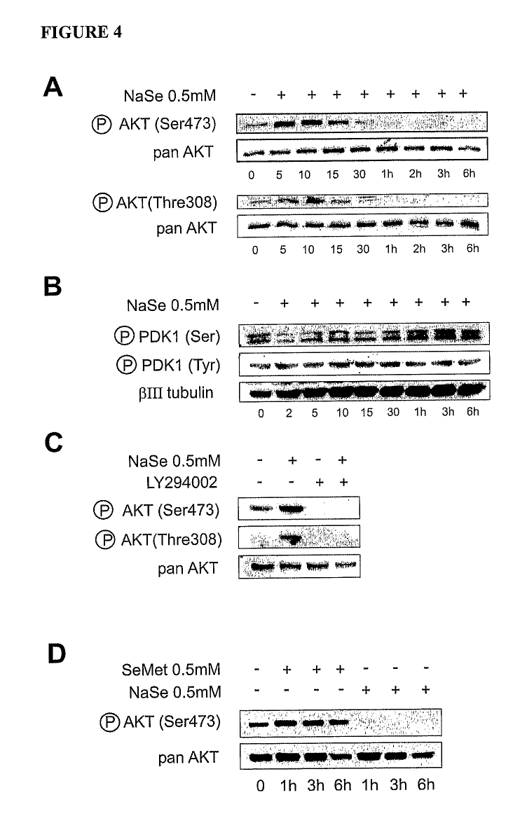

To determine whether sodium selenate can interfere with the activity of the PI3K pathway, PC-3 cells were treated with sodium selenate (0.5 mM) for various exposure times. The phosphorylation status of Akt in whole cell lysates using activation specific antibodies was determined. As shown in FIG. 4A, sodium selenate induced a transient boost in phosphorylation of Akt at Ser473 and Thr308 within 10 minutes of exposure to inorganic selenium. This boost was then followed by a marked and prolonged deactivation of Akt, whilst total cellular Akt levels (pan Akt) remained essentially unchanged (FIG. 3A). Akt is phosphorylated on Thr308 at the cellular membrane by PDK1 (Vanhaesebroeck, B. and Alessi, D. R. 2000, Biochem. J., 346 Pt3:561-576). To determine whether sodium selenate acted to downregulate PDK1 activity upstream of Akt, the phosphorylation status of PDK1 using phospho-specific PDK1 ser241 and tyr373/376 antibodies was analyzed. As shown in FIG. 4B, exposure of PC-3 cells to 0.5 mM sodium selenate out to 6 hours had no effect on the phosphorylation status of PDK1 indicating that sodium selenate does not inhibit Akt activation by blocking PDK1 kinase activity. To determine whether the transient boost in Akt activation observed with sodium selenate was dependent on upstream components of the PI3K pathway, PC-3 cells were treated with sodium selenate (0.5 mM) for 10 minutes, with or without pretreatment (1 hour) with the PI3K inhibitor LY294002 (50 μM). As observed in FIG. 4C, treatment with sodium selenate for 10 minutes boosted Akt activation as assessed by the phospho-specific antibodies. This boost was however completely blocked by pretreatment with LY294002, indicating that the transient boost in Akt phosphorylation induced by sodium selenate requires upstream components of the PI3K pathway.

The effects of inorganic selenium compounds on Akt activation in PC-3 cells were compared by treating cells with 0.5 mM sodium selenate or selenomethionine for various time points and assessing Akt phosphorylation at Ser473. As shown in FIG. 4D, selenomethionine was able to affect phosphorylation of Akt at all time periods measured, whilst sodium selenate profoundly inhibited Akt activation.

The results also indicated that sodium selenate inhibits downstream effectors of the cell survival, PI3K pathway. Potential effectors of PI3K signaling downstream of the PTEN tumor suppressor, include a number of substrates of the Akt kinase, such as BAD, caspase 9, IKKα and the Forkhead transcription factors FKHR, FKHRL1 and AFX (Biggs, et al 1999, Proc. Natl. Acad. Sci. USA, 96:7421-7426; Brunet et al 1999, Cell, 96:857-868; Cardone et al 1998, Science, 282:1318-1321. Datta et al 1997, Cell, 91:231-241; Kops et al 1999, Nature, 398:630-634; Ozes et al 1999, Nature, 401:82-85; Tang et al 1999, J. Biol. Chem., 274:16741-16746).

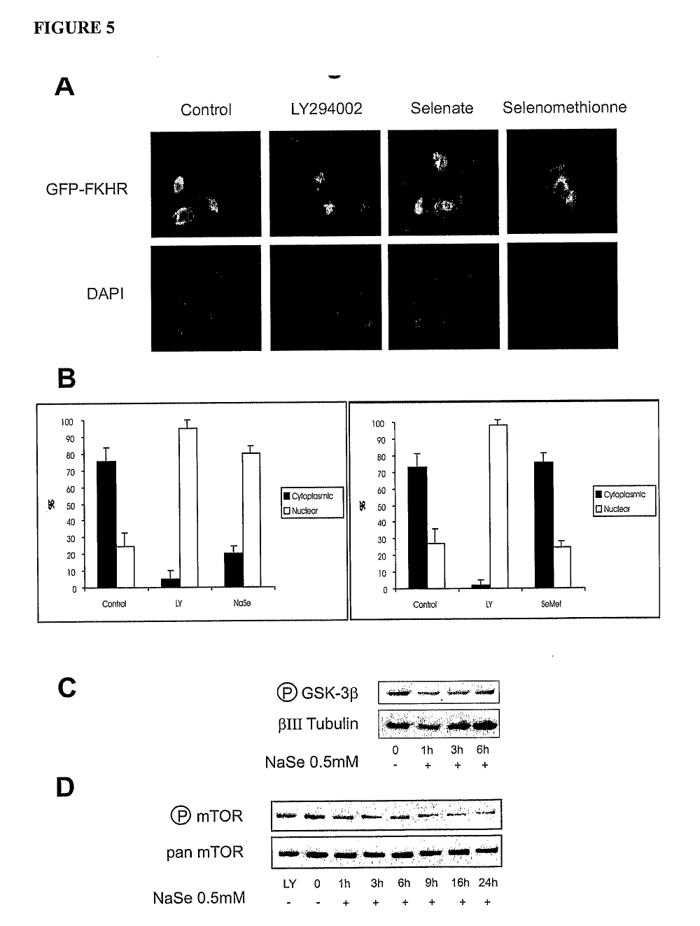

Members of the Forkhead transcription factor family are deregulated and inactive in PTEN-null cells, being aberrantly localized to the cytoplasm where they cannot activate transcription (Nakamura et al 2000, Mol. Cell. Biol., 20:8969-8982). Reintroducing PTEN function in such cells induces translocation of FKHR to the nucleus eventually causing G1 arrest in PTEN-null cells (Nakamura et al 2000, supra). Therefore, sodium selenate inhibition of Akt activation in PTEN-null PC-3 cells should similarly induce relocation of FKHR from the cytoplasm to the nucleus. PC-3 cells transfected with a GFP-FKHR expression construct were treated with LY294002 (50 μM, 1 hour) or sodium selenate (0.5 mM, 3 hours) or selenomethionine (0.5 mM, 3 hours) and the effects of the treatments on cellular localization of the fluorescent Forkhead protein were observed. As shown in FIGS. 5A and B the GFP-FKHR fusion protein was localized in the cytoplasm of untreated control PC-3 cells. Treatment with LY294002 or sodium selenate induced a marked relocalization of the GFP-FKHR fusion protein from the cytoplasm to the nucleus. Selenomethionine, however, was unable to induce relocalization of GFP-FKHR from the cytoplasm to the nucleus. These results confirm the selectivity between selenium compounds in blocking Akt signaling by inhibiting activation of the key Akt substrate protein, FKHR.

Two further downstream effectors/substrates of Akt are glycogen synthase kinase-3β, GSK-3β, (Moule et al 1997, J. Biol. Chem., 272:7713-7719; Van Weeren et al 1998, J. Biol. Chem., 273:13150-13156) and the mammalian target of rapamycin, mTOR protein (Nave et al 1999, Biochem. J., 344 Pt2:427-431; Sekulic et al 2000, Cancer Res., 60:3504-3513).

To determine whether sodium selenate could also effect activation of these Akt substrates, PC-3 cells were treated with sodium selenate (0.5 mM) for various time periods and probed whole cell lysates with phospho-specific antibodies to GSK-3β and mTOR. As shown in FIGS. 5C and 5D sodium selenate induced a decline in both mTOR and GAK-3β phosphorylation status.

### Discussion

The above results demonstrate the effects of high dose sodium selenate supplementation on hormone refractory prostate cancer. Sodium selenate was shown to inhibit PC-3 cell proliferation by inducing G1 arrest, in a dose- and time-dependent manner, but did not increase the level of apoptosis. The G1 arrest was accompanied by an increase in expression of cell cycle inhibitory proteins p27^{KIP1} and p21^{CIP1/WAF1} and by a decrease in expression of cyclin D1. Phosphorylation of Rb was also suppressed by selenate. Selenate, in contrast to selenomethionine, markedly decreased active levels of the PI3K effector, Akt. Activation of key downstream effectors of the PI3K/Akt pathway, mTOR, GSK-3β and the FKHR transcription factor were also inhibited by selenate, but not selenomethionine. These results demonstrate a specific inhibitory effect of inorganic selenate on the PI3K/Akt pathway, suggesting this compound may be of use in therapies for the treatment of cancer, particularly cancer caused by the over activation of the PI3K/Akt pathway, such as PTEN-null tumors.

The molecular mechanisms involved in the anti-tumorigenic effect of sodium selenate versus selenomethionine have also been elucidated in the present study. Inorganic sodium selenate inhibited tumor progression in vivo by impeding cell proliferation without having any effect on apoptosis. A dose- and time-dependent decrease in PC-3 cell proliferation in vitro was observed with sodium selenate able to inhibit S-phase progression of these cells by inducing G1 arrest without increasing the proportion of apoptotic cells observed by flow cytometry.

Progression through the cell cycle is controlled by CDKs, whose activity is inhibited by the CDK inhibitors. Progression through the G1-S transition is postulated to be controlled by the activity of G1 cyclins and CDKs. Cyclins such as cdk1 stimulate G1 cell cycle progression (Baldin et al 1993, Genes Dev., 7:812-821), whilst Rb appears to be a key downstream target coupling the cell cycle process to gene regulation (Weintraub et al 1995, Nature, 375:812-815). Phosphorylation of Rb by cdk4/cdk6/cyclin D complexes disrupts the Rb/E2F complex permitting E2F to activate genes required for DNA synthesis and cell cycle progression (Harbour et al 1999, Cell, 98:859-869).

The present results indicate that the levels of cyclin D1 decreased, as did the phosphorylation of Rb at Ser807/811, whereas cdk6 and cdk4 levels remained relatively unchanged. These results demonstrate that high dose selenate induces a G1 arrest in PC-3 cells by specifically interfering with the levels and activation of key cell cycle progression proteins. It is shown that the levels of p27^{KIP1} rose following exposure to high dose selenate, but not with high dose selenomethionine. Raised p27^{KIP1} levels are a hallmark and necessary for G1 arrest in a range of normal cells following serum deprivation (Coats et al 1996, Science, 272:877-880) or contact inhibition (Polyak et al 1994, Cell 78:59-66). p27^{KIP1} plays a central role as a negative regulator of cell cycle progression, hence raised levels of this protein provides a rationale for the observed G1 arrest induced by selenate. P21^{CIP1} expression also leads to G1 arrest by inhibiting cyclin/CDK complexes and by inhibiting DNA synthesis (Johnson, G. G. and Walker, C. L. 1999, Annu. Rev. Pharacol. Toxicol. 39:295-312).

The PI3K pathway has a central role in many cellular functions pertaining to proliferation and survival and the deregulation of this pathway, via loss of the PTEN tumor suppressor protein, is a common event in prostate malignancies (Whang et al 1998, Proc. Natl. Acad. Sci. USA 95:5246-5250). Activation of the PI3K pathway is required for the induction of cyclin D1 expression (Muise-Helmericks et al 1998, J. Biol. Chem. 273:29864-29872) and inhibition of this pathway with the PI3K specific inhibitor LY294002 has recently been reported to lead to G1 arrest in DU145 and PC-3 prostate carcinoma cells (Gao, et al 2003, Biochem. Biophys. Res. Commun. 310:1124-1132). A major downstream effector of the PI3K pathway is protein kinase B/Akt. Akt is constitutively activated in prostate tumors lacking PTEN, such as PC-3 cells. The present results indicate that high dose selenate but not selenomethionine could markedly decrease the levels of activated Akt present in these cells, an effect which acted specifically at the level of activated Akt as no change in PDK1 activation was detected, a kinase thought to directly induce activation of Akt downstream of PI3K (Vivanco, I. and Sawyers, C. L. 2003, supra). These results suggest that selenate may act to boost dephosphorylation of Akt, through an as yet poorly characterized phosphatase.

Interestingly, the effects of selenate on Akt activation appears to follow two distinct modes of action. Initially, selenate induces a boost in Akt phosphorylation. This boost is PI3K dependent as it is inhibited by pretreatment with LY294002. This transient boost is then followed by a long lasting, profound inhibition of Akt activation, which does not involve components upstream of Akt, as the proliferative inhibition of high dose sodium selenate is not enhanced by LY294002. Sodium selenate is also able to impede activation of the downstream Akt effectors, mTOR and GSK-3β, as well as the FKHR transcription factor, consistent with the inhibitory effect of selenate being focused on the key PI3K effector kinase Akt.

Methylseleninic acid also induces G1 arrest of DU-145 cells as well as apoptosis via a caspase-mediated pathway within 24 hours of treatment (Jiang et al 2001, supra). Doses of low as 3 μM induced G1 arrest but this was not associated with apoptosis or with decreased phosphorylation of Akt (Jiang et al 2002, supra). However, higher doses of 5 μM lead to a dose-dependent decline in Akt activation as well as initiation of caspase-mediated apoptosis. Inhibition of the PI3K pathway with LY294003 failed to induce apoptosis in DU-145 cells, indicating that the block in the PI3K pathway induced by methylseleninic acid was not involved in apoptosis induction (Jiang et al 2002, supra). Low dose Methylseleninic acid, inducing G1 arrest, independent of Akt inhibition and apoptosis at low dose but blocking Akt and inducing apoptosis at higher doses, contrasts sharply with the present results. Sodium selenate induced G1 arrest via inhibition of the PI3K pathway, specifically at the level of Akt, without inducing apoptosis. It would appear, therefore, that the anti-proliferative effects of selenate are more focused than those observed with other selenium compounds.

Based on the present findings and given that chronic activation of Akt is associated with resistance to chemotherapy in in vivo cancer models (Tanaka et al 2000, Oncogene, 19:5406-5412), an attractive mechanistic basis for the use of selenate with chemotherapeutic agents in combination therapy in tumor models is provided.

### Example 2

### REDUCTION OF PROSTATE TUMOR GROWTH DUE THE SYNERGISTIC EFFECT OF SODIUM SELENATE WITH PACLITAXEL

### Materials and Methods

1×10⁶ prostate tumor PC-3 cells were injected into the prostate of nude mice. Three days later 5 ppm selenium in the form of sodium selenate was administered to the drinking water of the mice receiving selenate. Paclitaxel at 10 mg/kg was administered intraperitoneally once per week in 10% Cremophor EL/25% ethanol/65% PBS solution to those animals receiving paclitaxel. 10 animals were used per group. After 5 weeks post injection, animals were sacrificed, the prostates removed, the appendages dissected off and the tumors weighed. The retroperitoneum was explored for lymphadenopathy and lymph nodes greater than 0.5 mm diameter excised. The tumors were then step sectioned at 50 micron steps and tumor volumes calculated from the digitized images using the standard volumetric formula (a +b²/2).

### Results

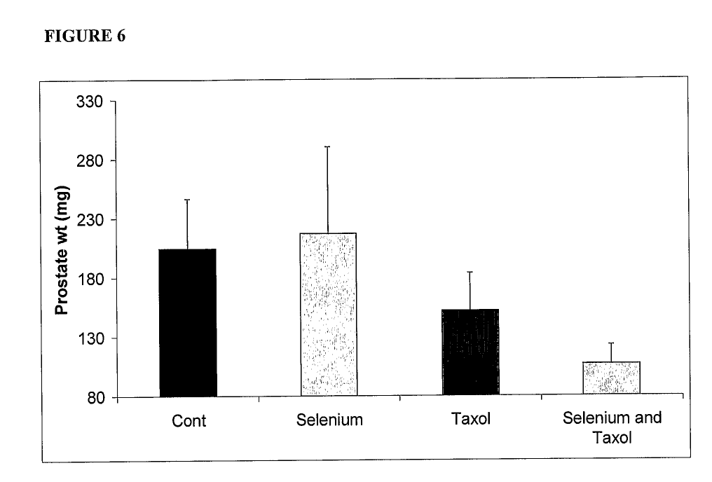

FIG. 6 demonstrates that selenate synergizes with paclitaxel to reduce prostate tumor weights. The control group received the paclitaxel solubilization carrier Cremophor and ethanol without paclitaxel. Cremophor is known to have some anti-tumor effect which probably accounts for the fact that the anti-tumor effect of selenate alone is not reduced compared to the control in this experiment. The results shown in FIG. 6 indicate that selenate and paclitaxel synergize to reduce tumor weights greater than the additive effects of selenate and paclitaxel alone. Selenate inhibits a key cell survival pathway, the PI3K/Akt pathway. This pathway is upregulated in a high proportion of human tumors and overactivation of this pathway is highly correlated with the development of chemoresistance to chemotherapeutic agents.

The present results indicate that co-treatment of prostate tumors in vivo with a combination therapy of selenate and paclitaxel can induce a greater anti-tumor effect than either treatment alone. Selenate could interfere with the tumor cells' ability to induce chemoresistance to the chemotherapeutic drug, paclitaxel. Therefore, prostate tumors were induced in nude mice by injecting PC-3 cells into the prostates of the animals and subsequently treating the animals with the selenate or paclitaxel alone or in combination for 5 weeks and then analyzing the tumors. As shown in FIG. 6, the combination therapy had a marked synergistic effect in reducing tumor weights greater than the additive effect of either treatment alone. The control group received the paclitaxel solubilization carrier Cremophor EL and ethanol without paclitaxel. Cremophor EL is known to have an anti-tumor effect which probably accounts for the fact that selenate alone is not reduced compared to the control in this experiment. The synergistic effects that are observed are greater in vivo than in vitro. This supports the concept that selenate is having an anti-angiogenic effect on vessel endothelial cells as well as on the growth of the tumor cells per se and that it is this combined effect only observable in vivo that accounts for the synergistic effect.

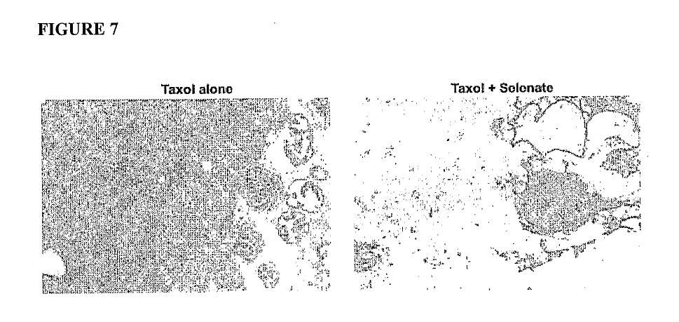

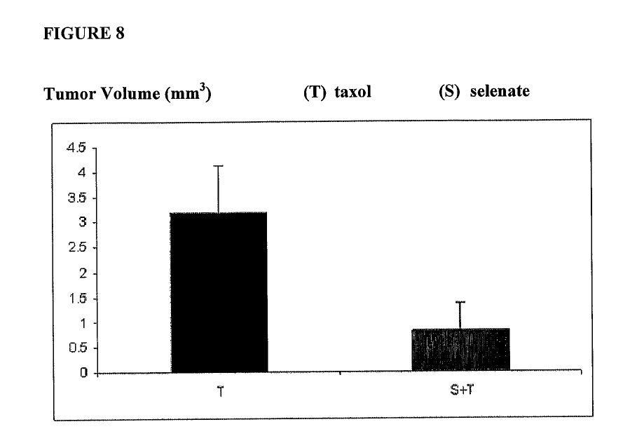

FIG. 7 shows tissue sections of tumors. It can be seen that selenate in combination with paclitaxel has a synergistic effect which significantly reduce the tumor size and volume compared to paclitaxel alone. This significant synergistic effect of selenate and paclitaxel is also evident in the graphs of tumor volumes indicated at FIG. 8.

The tumor volumes from paclitaxel alone treated and the combination therapy treated animals were measured to determine the effects of the agents on prostate tumor growth. The combination therapy had a marked effect on reducing tumor size as shown in FIG. 7, and also had a marked effect on tumor volumes (FIG. 8). This effect was statistically significant (P<0.05).

Therefore, the in vivo date shown in FIGS. 6 to 8 demonstrate the synergistic effect of selenate with paclitaxel to reduce prostate tumor weights and volumes. This effect was greater than the additive effects of either agent alone demonstrating the synergistic effects of the two compounds. This supports the finding that selenate is having an anti-angiogenic effect on vessel endothelial cells as well as on the growth of the tumor cells and that it is this combined effect observable in vivo that accounts for the synergistic effect. Selenate alone had a greater effect on reducing microvessel density in the tumors than on tumor volumes, indicating a direct anti-angiogenic effect.

### Example 3

### COMPARATIVE TESTS BETWEEN SELENATE AND SELENITE

### Materials and Methods

### Cell Toxicity

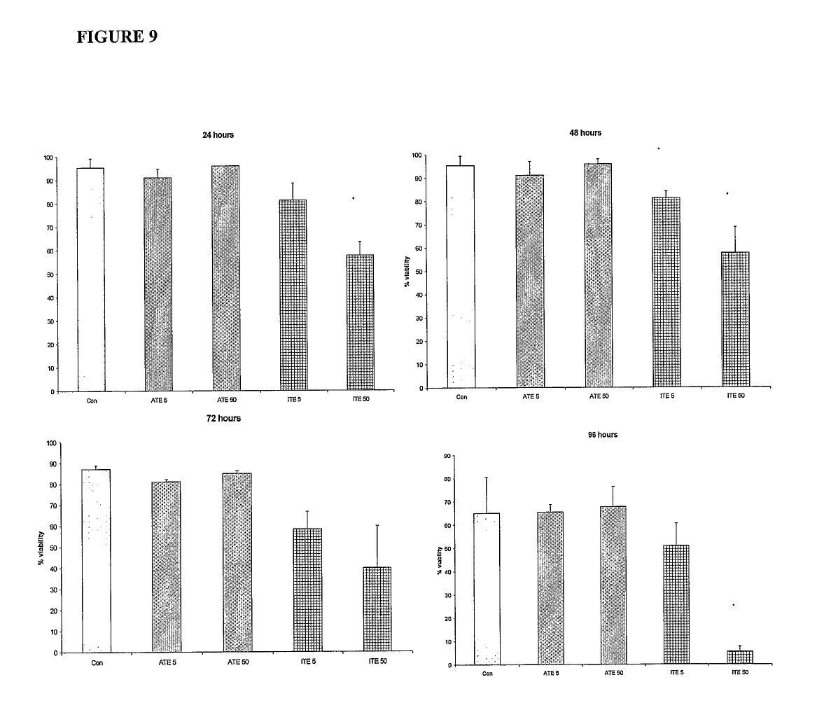

Cell Toxicity following treatment with 5 μM or 50 μM sodium selenate or sodium selenite involved measuring cell toxicity by Trypan Blue Exclusion. 5×10³ human prostate cancer PC-3 cells were seeded in a 24-well dish and 8 hours later were treated with either 5 μM or 50 μM of selenate or selenite. The dose range of 5 μM or 50 μM is equivalent to 0.1 mg/kg to 1.25 mg/kg. Cells were harvested at 24, 48, 72 and 96 hours following the addition of selenate or selenite and the percentage of viable cells as assessed by Trypan Blue staining was determined. The results shown in the graphs of FIG. 9 depict the means and SD of three independent experiments. FIG. 9 shows that selenate is cytostatic whereas selenite at similar concentrations is cytotoxic. Therefore, selenite would be unsuitable for combination therapy in animals.

In order to distinguish between the relative cytotoxic effects of selenate and selenite human prostate carcinoma cells, PC-3, were treated with two different concentrations of selenate at 5 μM or 50 μM (equivalent to a dose of 0.2 to 1.9 mg/kg) or selenite 5 μM or 50 μM (equivalent to a dose of 0.18 to 1.8 mg/kg). All cells in the tissue culture wells were then harvested sequentially at periods between 24 and 96 hours following addition of the treatments. The percentage of viable cells were then determined using the Trypan Blue exclusion assay. Selenate at all time points and at both the lower and higher concentration, was not cytotoxic to these cells. The percentages of viable cells in the selenate treated samples were equivalent to the control untreated samples (FIG. 9). This effect was maintained throughout all time points and reached statistical significance for a number of time points (see time points marked with asterisk, FIG. 9). Selenate has a demonstrated cytostatic effect on the cells whereas selenite has a cytotoxic effect.

### Cell Genotoxicity

5×10⁵ PC-3 cells were plated in a 6 well dish and 24 hours later cells were treated with paclitaxel at 1 μg/mL or 10 μg/mL or selenate 500 μM (19 mg/kg) or selenite 500 μM (18 mg/kg) for the indicated times. Cells were then washed in PBS, lysed in ELB lysis buffer and whole cell lysates run on a SDS-PAGE gel and blotted and probed with the indicated antibodies. Phosphorylation of Histone H2AX occurs within minutes of DNA damage, typically via DNA double or single strand breaks and is a very sensitive readout of DNA damage.

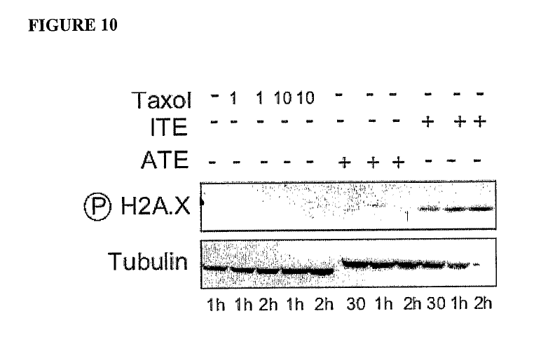

To ascertain the differences between selenate, selenite and the taxane, paclitaxel in inducing DNA damage PC-3 cells were treated with these compounds for increasing time periods then lysed. The protein extracts were probed for phosphorylation of Histone H2A.X using a phosphorylation specific antibody. The results are depicted in FIG. 10. FIG. 10 indicates that selenite is genotoxic, inducing DNA strand breaks whilst selenate and paclitaxel do not. Even at high (500 μM; equivalent to a dose of 19 mg/kg) concentration selenate, as well as increasing concentrations of paclitaxel did not induce DNA damage as assessed by Histone H2A.X phosphorylation. In contrast, selenite at the same concentration (500 μM; equivalent to a dose of 18 mg/kg) induces DNA damage within 30 minutes of treatment, an effect which increases with time in spite of the general toxic effects of this treatment as depicted by the concomitant decrease in β-tubulin levels in the same samples (FIG. 10).

### Activation of Akt

5×10⁵ PC-3 cells were plated in a 6-well dish and 24 hours later cells were serum starved for 16 hours then treated with the indicated selenium compounds all at 125 μM (equivalent to a dose range of 4-9 mg/kg) for 6 hours. Cells were then washed in PBS, lysed in ELB lysis buffer and whole cell lysates run on a SDS-PAGE gel and blotted and probed, firstly with an activation specific Akt antibody (Ser408), then the blots stripped and reprobed with a total Akt specific antibody (pan Akt) as a loading control. Signal intensities from the developed films were scanned and intensities of active Akt correlated to total Akt levels were plotted.

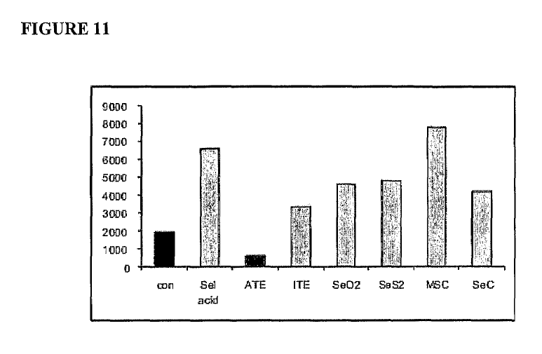

To compare the effects of different selenium compounds on the activation status of Akt, PC-3 cells were treated with seven different selenium compounds as shown in FIG. 11. FIG. 11 indicates that only sodium selenate (ATE) inhibits activation of Akt, reducing levels of phosphorylated Akt below control (con) levels. In contrast, Selenous acid (Sel acid), sodium selenite (ITE), selenium dioxide (SeO₂), selenium sulfide (SeS₂), Methyl selenocysteine (MSC), Selenocysteine (SeC) all induce activation of Akt above control (con) levels.

To determine the effects of high dose selenite, 5×10⁵ PC-3 cells were plated in a 6 well dish and 24 hours later cells were serum starved for 16 hours then treated with sodium selenite or sodium selenate at 500 μM (equivalent to a dose range of 18-19 mg/kg) for 1 hour. Cells were then washed in PBS, lysed in ELB lysis buffer and whole cell lysates run on a SDS-PAGE gel and blotted and probed firstly, with an activation specific Akt antibody (Ser408).

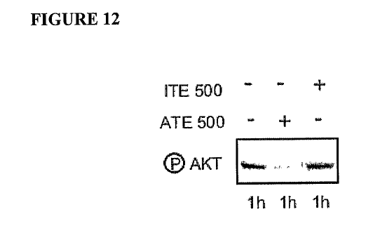

FIG. 12 indicates that even high dose sodium selenite (ITE) 500 μM does not inhibit Akt activation in comparison to a similar high dose of sodium selenate (ATE). To determine whether high dose levels of selenite would be able to inhibit Akt activation levels similar to that observed with selenate, PC-3 cell were treated with 500 μM (18-19 mg/kg) sodium selenate or sodium selenite for 1 hour and activation of Akt determined using a phosphorylation specific antibody. This time period was chosen because selenite at 500 μM (18 mg/kg) does not induce general protein degradation at this time point. As shown in FIG. 12, selenate inhibited activation of Akt compared to control untreated cells whilst selenite was unable to inhibit activation of Akt.

### Apoptosis

5×10⁵ PC-3 cells were plated in a 6 well dish and 24 hours later cells were treated with paclitaxel at 1, 10, 100 ng/mL and 1 μg/mL, 101 μg/mL or sodium selenate at 100 μM, 250 μM or 500 μM (equivalent to 4 to 19 mg/kg) or sodium selenite at 100 μM, 250 μM or 500 μM (equivalent to 3.6 to 18 mg/kg) for 16 hours. Cells were then wash in PBS, lysed in ELB lysis buffer and whole cell lysates run on a SDS-PAGE gel and blotted and probed with anti-cleaved PARP (PARP) and anti-β-tubulin (tubulin) specific antibodies.

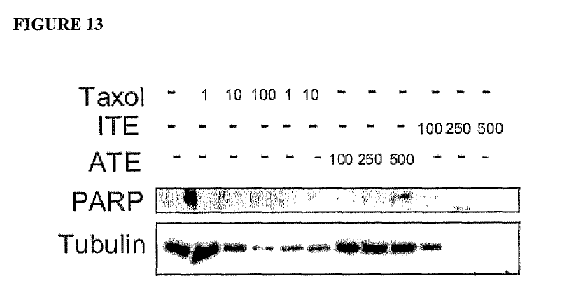

Agents that induce genotoxic damage elicit apoptotic programs via p53 dependent mechanisms as has been demonstrated for selenite (Jiang, C. et al., 2004 Mol. Can. Ther. 3:877). Microtubule stabilizing agents such as paclitaxel induce apoptosis via the intrinsic apoptotic pathway. To differentiate the pro-apoptotic mechanisms of selenate and selenite PC-3 cells were treated with increasing concentrations of these compounds for 16 hours and analyzed levels of the apoptotic marker protein, cleaved PARP. FIG. 13 indicates that selenate and selenite induce apoptosis through different mechanisms. Paclitaxel and selenate induce cleavage of the pro-apoptotic PARP protein whilst selenite does not. Selenite also induces marked degradation of cellular-tubulin underlying its cellular toxicity. Therefore, selenate and selenite appear to induce apoptosis via different mechanisms, as evidence by their effects on PARP and tubulin the overall data show a clear differentiation in the effect of various selenium compounds on the Akt pathway.

### Example 4

5×10⁴ human prostate cancer androgen sensitive LNCaP cells were seeded in a 6-well dish and 8 hours later were treated with 50 μM sodium selenate or not (control). Cells were harvested at 72 hours following addition of the compounds and cell counts of viable cells as assessed by Trypan Blue staining were determined.

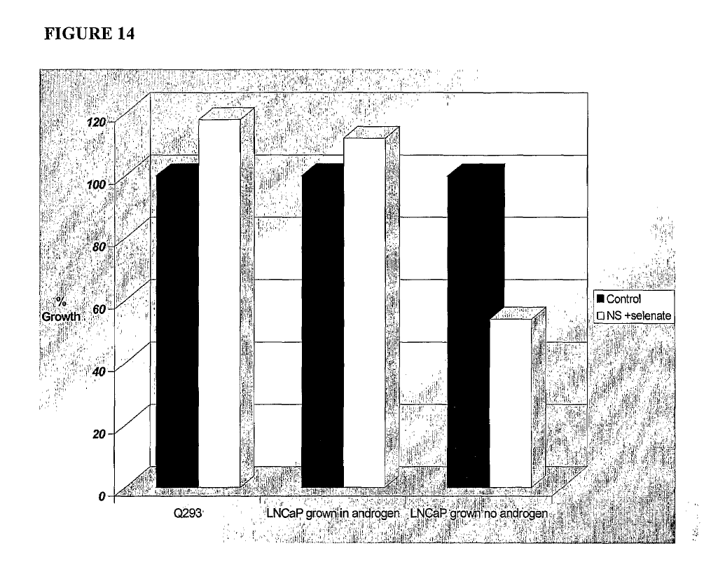

As shown in FIG. 14, selenate synergizes with androgen ablation to reduce parental LNCaP cell proliferation after 72 hours of treatment.

### Example 5

5×10⁴ human prostate cancer androgen independent CSS LNCaP cells were seeded in a 6-well dish and 8 hours later were treated with either 50 μM selenate or not (control). Cells were harvested at 72 hours following addition of the sodium selenate and cell counts of viable cells as assessed by Trypan Blue staining were determined.

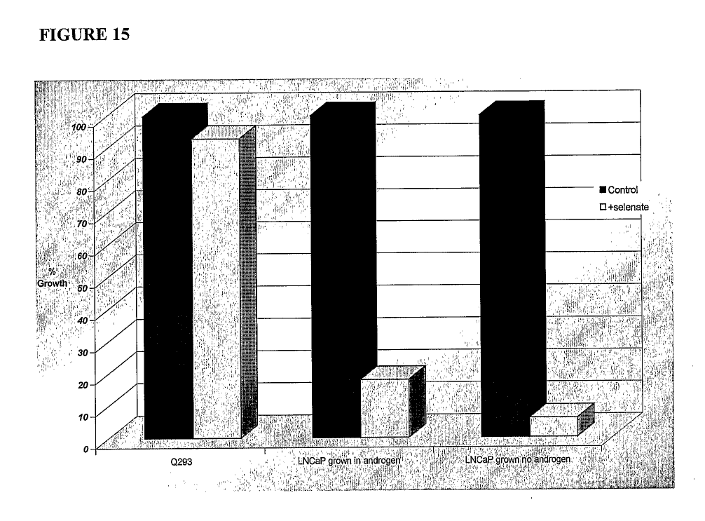

As shown in FIG. 15, selenate synergizes with androgen ablation to reduce CSS LNCaP cell proliferation after 72 hours of treatment. Androgen independent cells are even more sensitive to selenate treatment than parental LNCaP cells.

### Example 6

1×10⁵ human prostate cancer androgen sensitive NS LNCaP cells were seeded in a 6-well dish and 8 hours later were treated with either 5 μM sodium selenate or 10 μM LY294002. Cells were harvested at the indicated periods following addition of the sodium selenate or Ly294002 and cell counts of viable cells as assessed by Trypan Blue staining were determined.

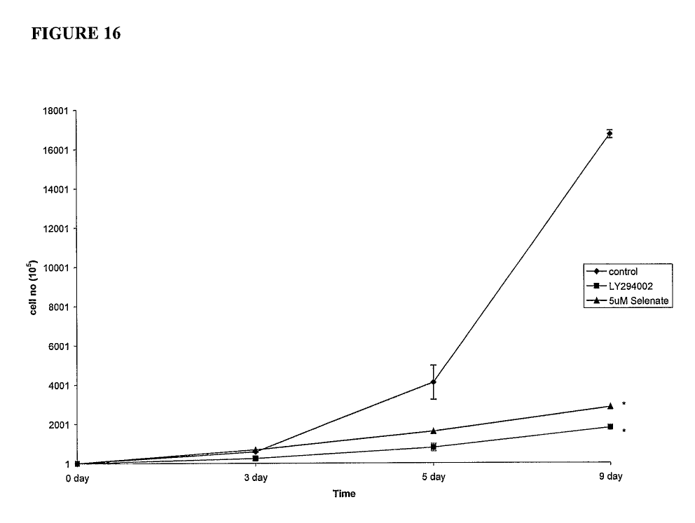

As shown in FIG. 16, selenate at only 5 μM markedly reduces (p<0.95) androgen sensitive NS LNCaP cell proliferation after 9 days of treatment.

### Example 7

1×10⁵ human prostate cancer androgen sensitive NS LNCaP cells were seeded in a 6-well dish and 8 hours later were treated with either 5 μM sodium selenate or 10 μM LY294002. Cells were harvested at the indicated periods following addition of the compounds and cell counts of viable cells as assessed by Trypan Blue staining were determined.

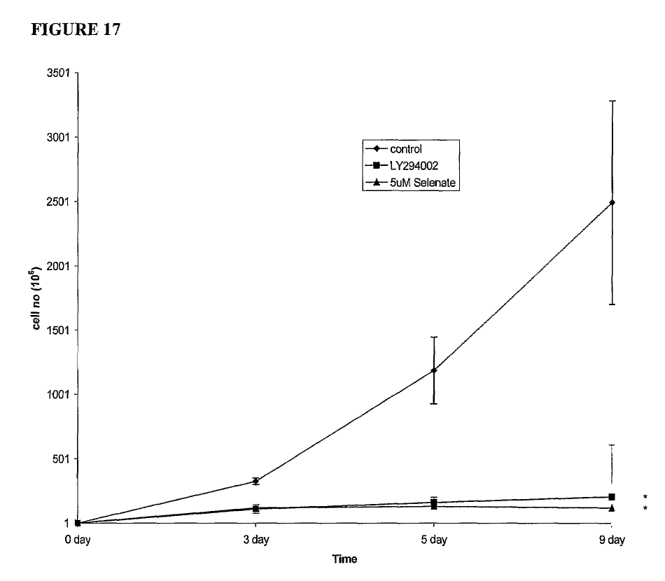

As shown in FIG. 17 selenate at only 5 μM synergizes with androgen ablation to markedly reduce (p<0.95) androgen sensitive NS LNCaP cell proliferation after 9 days of treatment.

### Example 8

1×10⁵ human prostate cancer androgen independent CSS LNCaP cells were seeded in a 6-well dish and 8 hours later were treated with either 5 μM sodium selenate or 10 μM LY294002. Cells were harvested at the indicated periods following addition of the sodium selenate or LY294002 and cell counts of viable cells as assessed by Trypan Blue staining were determined.

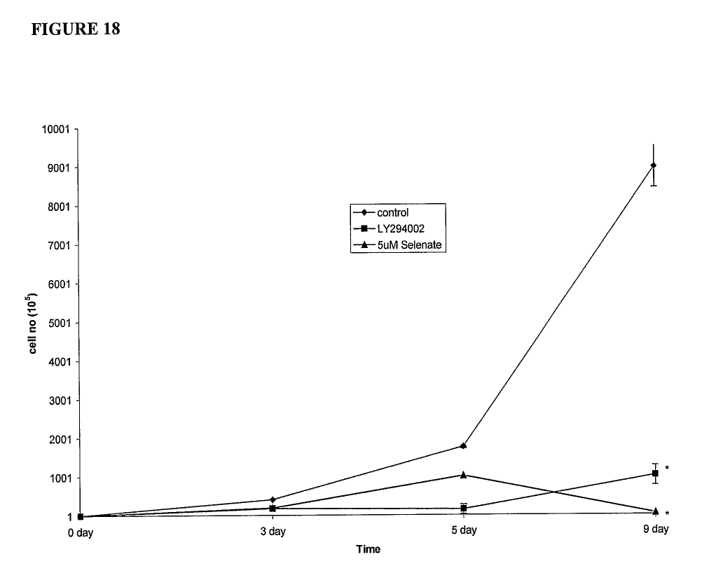

As shown in FIG. 18 selenate at only 5 μM markedly reduces (p<0.95) androgen independent CSS LNCaP cell proliferation after 9 days of treatment, even in the presence of testosterone.

### Example 9

1×10⁵ human prostate cancer androgen independent CSS LNCaP cells were seeded in a 6-well dish and 8 hours later were treated with either 5 μM sodium selenate or 10 μM LY294002. Cells were harvested at the indicated periods following addition of the sodium selenate or LY294002 and cell counts of viable cells as assessed by Trypan Blue staining were determined.

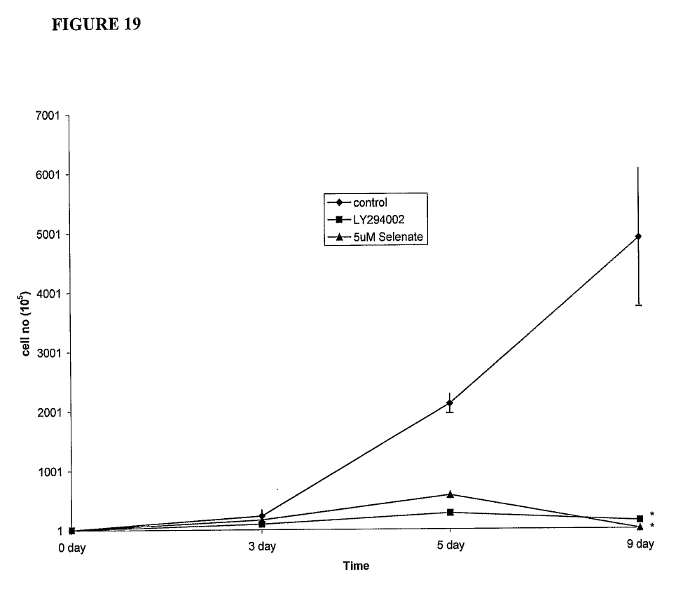

As shown in FIG. 19, selenate at only 5 μM synergizes with androgen ablation to markedly reduce (p<0.95) androgen sensitive CSS LNCaP cell proliferation after 9 days of treatment.

### Example 10

1×10⁵ human kidney epithelial Q293 cells were seeded in a 6-well dish and 8 hours later were treated with either 5 μM sodium selenate or 10 μM LY294002. Cells were harvested at the indicated periods following addition of the sodium selenate or LY294002 and cell counts of viable cells as assessed by Trypan Blue staining were determined.

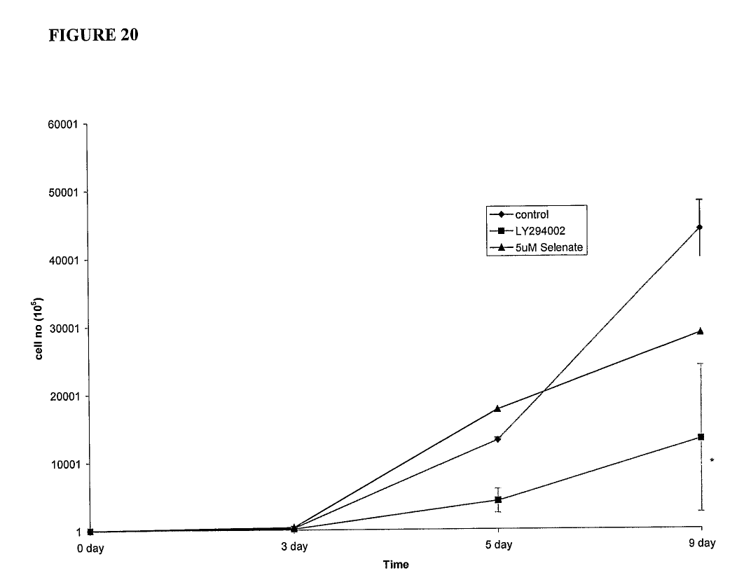

As shown in FIG. 20, selenate at 5 μM does not significantly effect Q293 cell proliferation whereas the PI3K inhibitor LY294002 does significantly effect proliferation (p<0.95) after 9 days of treatment. Selenates inhibitory effect is therefore cell specific.

### Example 11

1×10⁵ human kidney epithelial Q293 cells were seeded in a 6-well dish and 8 hours later were treated with either 5 μM sodium selenate or 10 μM LY294002. Cells were harvested at the indicated periods following addition of the sodium selenate or LY294002 and cell counts of viable cells as assessed by Trypan Blue staining were determined.

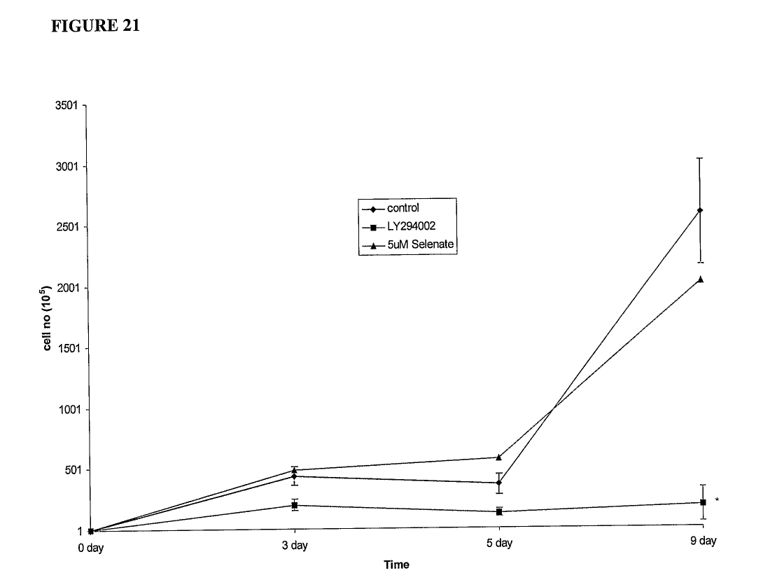

As shown in FIG. 21, selenate at 5 μM does not significantly effect Q293 cell proliferation even when combined with androgen ablation (grown in CSS media) whereas the PI3K inhibitor LY294002 does significantly effect proliferation (p<0.95) after 9 days of treatment. Selenates inhibitory effect is therefore cell specific.

### Materials And Methods For Examples 4 To 11

### Cell Culture

The parental androgen-sensitive LNCaP cell line was obtained from the American Type Culture Collection (Manassas, Va., USA) and routinely cultured in RPMI 1641 (Invitrogen) supplemented with 10% foetal calf serum and 1% antibiotic/antimycotic mixture (Invitrogen). Cells were maintained at 37° C. in 5% CO₂. To select androgen independent LNCaP cells LNCaPs were cultured in RPMI 1641 (Invitrogen) in 5% Charcoal stripped serum (CSS) (Invitrogen) which has removed all detectable traces of testosterone, for 6-8 weeks and obtained LNCaP cell lines which were now able to freely proliferate in the absence of testosterone. These cells are referred to as CSS LNCaPs.

Q293 cells are a human kidney epithelial cell line routinely cultured in DMEM media (Invitrogen) in 5% foetal calf serum and 1% antibiotic/antimycotic mixture (Invitrogen).

Sodium selenate was made up as 10 mM stock solutions in distilled water and filter sterilized before dilution in media for in vitro experiments. The PI3K inhibitor, LY294002 was dissolved in DMSO at 50 mM stock solution and diluted in cell culture media for in vitro experiments.

### Cell Growth Curve

Between 5×10⁴ (FIGS. 1-2) and 1×10⁵ LNCaP, CSS LNCaP or Q293 cells were allowed to attach overnight. After some hours the media was changed to include sodium selenate or LY294002 in the presence of either normal serum or charcoal stripped serum (CSS) as indicated and allowed to grow until the specified time points. Supernatants and cells were then harvested, combined and viable cells assessed by Trypan Blue exclusion assay. Experiments were performed in triplicate.

### Statistical Analysis

Data are presented as mean ± SE. unless otherwise indicated. Differences between treatments and the control group were analyzed using pairwise t test with significance assumed at p<0.05. Asterisks represent values significantly different from the corresponding control value. Statistical analysis was performed using SPSS 9.05 for Windows (SPSS, Chicago, Ill.) software.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method for inhibiting the growth of a tumor cell in which the Akt signaling pathway is activated, the method comprising exposing the tumor cell to an Akt signaling pathway activation-inhibiting amount of selenate or a pharmaceutically acceptable salt thereof.

2. The method according to claim 1, wherein the tumor cell is one in which Akt is over-active.

3. The method according to claim 1, wherein the tumor cell is a prostate tumor cell.

4. The method according to claim 3, wherein the growth of the tumor cell is androgen independent or chemoresistant.

5. The method according to claim 1, wherein the Akt signaling pathway activation-inhibiting amount of selenate or its pharmaceutically acceptable salt is from about 0.015 mg/kg to about 20.0 mg/kg.

6. The method according to claim 1, further comprising exposing the tumor cell to a cytostatic agent or cytotoxic agent.

7. The method according to claim 6, wherein the cytostatic agent is a microtubule stabilizing agent.

8. The method according to claim 7, wherein the microtubule stabilizing agent is paclitaxel.

9. The method according to claim 1, further comprising exposing the tumor cells to radiotherapy, optionally together with a radiosensitizing agent.

10. A method for treating a cancer in which the Akt signaling pathway is activated, the method comprising administering to a subject in need of such treatment an Akt signaling pathway activation-inhibiting amount of selenate or a pharmaceutically acceptable salt thereof.

11. The method according to claim 10, wherein the cancer is one in which Akt is overactive.

12. The method according to claim 10, wherein the cancer is prostate cancer.

13. The method according to claim 12, wherein the cancer is an androgen-independent prostate cancer or a chemoresistant prostate cancer.

14. The method according to claim 10, wherein the Akt signaling pathway activation-inhibiting amount of selenate is a supranutritional amount.

15. The method according to claim 10, wherein the Akt signaling pathway activation-inhibiting amount of selenate is from about 0.015 mg/kg to about 20.0 mg/kg.

16. The method according to claim 10, wherein the selenate is in the form of sodium selenate.

17. The method according to claim 10, further comprising administering a cytostatic agent or a cytotoxic agent.

18. The method according to claim 17, wherein the cytostatic agent is a microtubule stabilizing agent.

19. The method according to claim 18, wherein the microtubule stabilizing agent is paclitaxel.

20. The method according to claim 10, further comprising administration of radiotherapy, optionally in combination with a radiosensitizing agent.

21. A method for treating a hormone-dependent cancer in a subject, the method comprising administering a therapeutically effective amount of selenate or a pharmaceutically acceptable salt thereof in combination with hormone ablation therapy.

22. The method according to claim 21, wherein the therapeutically effective amount of selenate or its pharmaceutically acceptable salt is a supranutritional amount.

23. The method according to claim 21, wherein the therapeutically effective amount of selenate or its pharmaceutically acceptable salt is from about 0.015 mg/kg to about 20 mg/kg.

24. The method according to claim 21, wherein the selenate is in the form of sodium selenate.

25. The method according to claim 21, wherein the hormone-dependent cancer is selected from the group consisting of an androgen-dependent cancer or an estrogen-dependent cancer.

26. The method according to claim 21, wherein the hormone-dependent cancer is selected from the group consisting of prostate cancer, testicular cancer, breast cancer, ovarian cancer, uterine cancer, endometrial cancer, thyroid cancer of and pituitary cancer.

27. The method according to claim 21, wherein the hormone-dependent cancer is an androgen-dependent prostate cancer.

28. The method according to claim 21, further comprising administration of a cytostatic agent or a cytotoxic agent.

29. The method according to claim 28, wherein the cytostatic agent is a microtubule stabilizing agent.

30. The method according to claim 29, wherein the microtubule stabilizing agent is paclitaxel.

31. The method according to claim 21, further comprising administration of radiotherapy, optionally together with a radiosensitizing agent.

32. A method for treating prostate cancer in a subject, the method comprising administering to the subject a therapeutically effective amount of selenate or a pharmaceutically acceptable salt thereof.

33. The method according to claim 32, wherein the prostate cancer is an androgen-independent prostate cancer or a chemoresistant prostate cancer.

34. The method according to claim 32, wherein the therapeutically effective amount is a supranutritional amount.

35. The method according to claim 32, wherein the therapeutically effective amount of selenate or its pharmaceutically acceptable salt thereof is from about 0.015 mg/kg to about 20.0 mg/kg.

36. The method according to claim 32, wherein the selenate is in the form of sodium selenate.

37. The method according to claim 32, further comprising administering a cytostatic agent or a cytotoxic agent.

38. The method according to claim 37, wherein the cytostatic agent is a microtubule stabilizing agent.

39. The method according to claim 38, wherein the microtubule stabilizing agent is paclitaxel.

40. A method according to claim 32, further comprising administering radiotherapy, optionally in combination with a radiosensitizing agent.

41. (canceled)

42. (canceled)

43. (canceled)

44. A pharmaceutical composition comprising selenate or a pharmaceutically acceptable salt thereof and at least one cytostatic or cytoxic agent and a pharmaceutically acceptable carrier.

45. The pharmaceutical composition according to claim 44, wherein the cytostatic agent is selected from the group consisting of a microtubule-stabilizing agent, a kinase inhibitor, a receptor kinase targeted antibody, an mTOR pathway inhibitor, an Ap02L/Trail antiangiogenic agent, an antineoplastic immunotherapy vaccine, an antibiotic cytotoxic agent, an alkylating agent, a hormonal antineoplastic agent, a gonadal hormone, an antimetabolite, an anabolic agent, an adrenal steroid hormone, a neoplastic agent and a topoisomerase inhibitor.

46. The pharmaceutical composition according to claim 45, wherein the cytostatic agent is a microtubule-stabilizing agent.

47. The pharmaceutical composition according to claim 46, wherein the microtubule-stabilizing agent is selected from the group consisting of taxanes, paclitaxel, docetaxel, epothilones and laulimalides.

48. The pharmaceutical composition according to claim 44, wherein the cytotoxic agent is selected from the group consisting of anthracyclines, CMF agents, cisplatin, carboplatin, bleomycin, topotecan, irinotecan, melphalan, chlorambucil, vincristine, vinblastine and mitomycin-C.

49. The pharmaceutical composition according to claim 44 further comprising a hormone ablation agent.

50. The pharmaceutical composition according to claim 44, further comprising a radiosensitising agent.

51. A pharmaceutical composition comprising selenate or a pharmaceutically acceptable salt thereof and a hormone ablation agent and a pharmaceutically acceptable carrier.

52. The pharmaceutical composition according to claim 51 wherein the hormone ablation agent is selected from the group consisting of GnRH agonists or antagonists, agents that interfere with the androgen receptor, and agents that interfere with steroid biosynthesis.

53. The pharmaceutical composition according to claims 51, further comprising a radiosensitising agent.
